(19) United States
(12) Patent Application Publication
(10) Pub. No.: US 2024/0145091 A1
(43) Pub. Date: May 2, 2024

(54) METHOD FOR MODULATING THE AMOUNT OF VIRUS, METHOD FOR TREATING OR PREVENTING VIRUS INFECTIONS DISEASE, METHOD FOR ESTIMATING THE AMOUNT OF VIRUS, AND METHOD FOR PREDICTING THE PROGNOSIS OF VIRUS INFECTION DISEASE IN SUBJECT, AS WELL AS COMPOSITION AND SYSTEM FOR THE SAME

(71) Applicants: National Institutes of Biomedical Innovation, Health and Nutrition, Osaka (JP); Kagami Inc., Osaka (JP)

(72) Inventors: Tomonori KIMURA, Osaka (JP); Shihoko KIMURA, Osaka (JP); Yasuhiro YASUTOMI, Ibaraki (JP); Masamitsu ASAKA, Ibaraki (JP); Emiko URANO, Ibaraki (JP); Daichi UTSUMI, Ibaraki (JP); Masashi MITA, Osaka (JP)

(73) Assignees: National Institutes of Biomedical Innovation, Health and Nutrition, Osaka (JP); Kagami Inc., Osaka (JP)

(21) Appl. No.: 18/488,380

(22) Filed: Oct. 17, 2023

(30) Foreign Application Priority Data
Oct. 18, 2022 (JP) 2022-167239

(51) Int. Cl. G16H 50/20; A61K 31/198; A61K 31/401; A61P 31/12; G16H 20/10

(52) U.S. Cl. CPC G16H 50/20; A61K 31/198; A61K 31/401; A61P 31/12; G16H 20/10

(57) ABSTRACT

Provided herein is a method for modulating the amount of virus in a subject comprising administering to the subject an agent for controlling an indicator associated with a D-amino acid in the subject.

### FIELD

The present invention relates to a method for modulating the amount of virus in a subject, a method for treating or preventing virus infection disease in a subject, a method for estimating the amount of virus in a subject, and a method for predicting the prognosis of virus infection disease in a subject, as well as a composition and a system for these methods.

### BACKGROUND

Infection is defined as the establishment, invasion, and multiplication of a pathogen (pathogenic microorganism) such as a virus in cells, tissues, and organs of a host through various infection routes. Pathogens that invade the host are eliminated from the host by the host's defense mechanism in most cases, but when infection is established and leads to occurrence of some symptoms or signs such as fever (onset of disease), the condition is referred to as infection disease. Infections caused by viruses such as coronaviruses and influenza viruses and the resulting infection diseases can pose a threat to healthcare and the economy through pandemics. Genetic tests (e.g., PCR method), immunological tests (e.g., antigen detection method), antibody/antigen tests, etc. have been put into practice for the detection of the presence of a virus and its quantity in the host. Urinary liver-type fatty acid-binding protein (L-FABP) has been proposed as a biomarker for predicting the risk of increased severity of viral infections (Patent Literature 1).

In recent years, advances in the performance of techniques for identifying and analyzing chiral amino acids have led to the development of quantitative studies that identify trace amounts of D- and L-amino acids in mammals and other living organisms. This has clarified the existence and functions of some D-amino acids, which have conventionally been treated as total amino acids (D-amino acids+L-amino acids) or L-amino acids for convenience due to technical limitations. In the mammalian intestine, host intestinal immunity has been shown to be regulated via D-amino acid metabolism by D-amino acid oxidase (DAO) against Vibrio spp. (Non-Patent Literature 1). It has also been reported that the % D values ({(D-amino acid)/(D-amino acid+L-amino acid)}×100) of D-asparagine, D-serine, D-alanine, and D-proline in blood of humans infected with human immunodeficiency virus (HIV) and receiving antiretroviral therapy correlated with the age and renal function markers (eGFR) of subjects, but that no variation was observed with HIV infection (Non-patent Literature 2).

### CITATION LIST

### Patent Literature

[Patent Literature 1] JP 6933834 B
[Patent Literature 2] JP 6868878 B
[Patent Literature 3] JP 6993654 B
[Patent Literature 4] WO 2020/196436 A
[Patent Literature 5] WO 2013/140785 A

### Non-Patent Literature

[Non-Patent Literature 1] Sasabe J, Miyoshi Y, Rakoff-Nahoum S, Zhang T, Mita M, Davis B M, Hamase K, Waldor M K. Interplay between microbial D-amino acids and host D-amino acid oxidase modifies murine mucosal defence and gut microbiota. Nat Microbiol. 2016 Jul. 25; 1(10):16125. doi: 10.1038/nmicrobio1.2016.125.
[Non-Patent Literature 2] Yap S H, Lee C S, Furusho A, Ishii C, Shaharudin S, Zulhaimi N S, Kamarulzaman A, Kamaruzzaman S B, Mita M, Leong K H, Hamase K, Rajasuriar R. Plasma D-amino acids are associated with markers of immune activation and organ dysfunction in people with HIV. AIDS. 2022 Jun. 1; 36(7):911-921. doi: 10.1097/QAD.0000000000003207.
[Non-Patent Literature 3] Kawamura M, Hesaka A, Taniguchi A, Nakazawa S, Abe T, Hirata M, Sakate R, Horio M, Takahara S, Nonomura N, Isaka Y, Imamura R, Kimura T. Measurement of glomerular filtration rate using endogenous D-serine clearance in living kidney transplant donors and recipients. EClinicalMedicine. 2021 Dec. 5; 43:101223. doi: 10.1016/j.eclinm.2021.101223.
[Non-Patent Literature 4] Hesaka A, Yasuda K, Sakal S, Yonishi H, Namba-Hamano T, Takahashi A, Mizui M, Hamase K, Matsui R, Mita M, Horio M, Isaka Y, Kimura T. Dynamics of D-serine reflected the recovery course of a patient with rapidly progressive glomerulonephritis. CEN Case Rep. 2019 November; 8(4):297-300. doi: 10.1007/s13730-019-00411-6.
[Non-Patent Literature 5] Sasabe J, Miyoshi Y, Suzuki M, Mita M, Konno R,

Matsuoka M, Hamase K, Aiso S. D-amino acid oxidase controls motoneuron degeneration through D-serine. Proc Natl Acad Sci USA. 2012 Jan. 10; 109(2):627-32. doi: 10.1073/pnas.1114639109.
[Non-Patent Literature 6] Miyoshi Y, Konno R, Sasabe J, Ueno K, Tojo Y, Mita M, Aiso S, Hamase K. Alteration of intrinsic amounts of D-serine in the mice lacking serine racemase and D-amino acid oxidase. Amino Acids. 2012 November; 43(5):1919-31. doi: 10.1007/s00726-012-1398-4.
[Non-Patent Literature 7] Hesaka A, Tsukamoto Y, Nada S, Kawamura M, Ichimaru N, Sakal S, Nakane M, Mita M, Okuzaki D, Okada M, Isaka Y, Kimura T. D-Serine mediates cellular proliferation for kidney remodeling. Kidney360. 2021 Aug. 16; 2(10):1611-1624. doi: 10.34067/KID.0000832021.
[Non-Patent Literature 8] Wiriyasermkul P, Moriyama S, Tanaka Y, Kongpracha P, Nakamae N, Suzuki M, Kimura T, Mita M, Sasabe J, Nagamori S. D-Serine, an emerging biomarker of kidney diseases, is a hidden substrate of sodium-coupled monocarboxylate transporters. bioRxiv. 2020. doi: 10.1101/2020.08.10.244822.
[Non-Patent Literature 9] Asaka M N, Utsumi D, Kamada H, Nagata S, Nakachi Y, Yamaguchi T, Kawaoka Y, Kuba K, Yasutomi Y. Highly susceptible SARS-CoV-2 model in CAG promoter-driven hACE2-transgenic mice. JCI Insight. 2021 Oct. 8; 6(19):e152529. doi: 10.1172/jci.insight.152529.

### SUMMARY

During normal life activities, the balances of D-amino acids in the body are controlled within a certain range. Variations in the D-amino acid balances associated with viral infections or changes in the amount of virus in a subject may increase the risk of health problems. It is therefore necessary to correct and adjust the balances.

The present inventors have comprehensively and precisely quantified and analyzed chiral amino acids (amino acids identified as D-amino acids and L-amino acids) in blood of virus-infected subjects, and have found that the amounts of D-amino acids in the blood of virus-infected subjects shifted (decreased and increased), and that these shifts related to the amount of virus in the subject and the symptoms and pathology of the virus infection disease. Furthermore, as a result of intensive research on the effects and mechanisms when the amounts of D-amino acids were artificially shifted in the subject's body, the present inventors have developed a technique to suppress the aggravation of virus infection disease and improve the prognosis by adjusting the amount of virus in the subject through modulating the amounts of D-amino acids in the body, and have thus completed the present invention that provides a solution to the above problem.

Embodiments of the present invention relate to, e.g., the following aspects.

### [Aspect 1]

A method for modulating the amount of virus in a subject, comprising:

administering to the subject an agent for controlling an indicator associated with a D-amino acid in the subject.

### [Aspect 2]

The method according to Aspect 1, wherein the indicator associated with a D-amino acid is a measurement value for the D-amino acid in blood or its correction value or correction formula.

### [Aspect 3]

The method according to Aspect 1 or 2, wherein the agent for controlling an indicator associated with a D-amino acid in the subject is a D-amino acid.

### [Aspect 4]

The method according to any one of Aspects 1 to 3, wherein the D-amino acid is one or more D-amino acids selected from the group consisting of D-proline, D-serine, D-alanine, and D-asparagine.

### [Aspect 5]

The method according to any one of Aspects 1 to 4, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

### [Aspect 6]

The method according to any one of Aspects 1 to 5, further comprising treating or preventing virus infection in the subject by modulating an indicator associated with a D-amino acid in the subject which has been shifted due to virus infection.

### [Aspect 7]

The method according to Aspect 6, wherein the shift in the indicator associated with a D-amino acid in the subject due to virus infection is a shift indicating a decrease in the amount of the D-amino acid in blood.

### [Aspect 8]

The method according to Aspect 6 or 7, wherein the modulating of the indicator associated with a D-amino acid in the subject includes supplementing the D-amino acid in blood which has been decreased due to virus infection.

### [Aspect 9]

A method for estimating the amount of virus in a subject, comprising:

making a determination on the amount of virus in the subject using an indicator associated with a D-amino acid in the subject.

### [Aspect 10]

The method according to Aspect 9, wherein when the indicator associated with a D-amino acid indicates that the amount of a D-amino acid in blood has decreased or is equal to or lower than a predetermined threshold, it is determined that the amount of virus in the subject is increasing.

### [Aspect 11]

The method according to Aspect 9 or 10, wherein the indicator associated with a D-amino acid indicates that the amount of a D-amino acid in blood has ceased to decrease or has increased, it is determined that the amount of virus in the subject has ceased to increase or is decreasing.

### [Aspect 12]

The method according to any one of Aspects 9 to 11, further comprising:

predicting the prognosis of virus infection disease in the subject based on the results of determination on the amount of virus in the subject.

### [Aspect 13]

The method according to Aspect 12, wherein when the indicator associated with a D-amino acid indicates that the amount of a D-amino acid in blood has decreased or is equal to or lower than a predetermined threshold, the prognosis of the subject is determined to be aggravation or increase in severity of virus infection disease.

### [Aspect 14]

The method according to Aspect 12 or 13, wherein the indicator associated with a D-amino acid indicates that the amount of a D-amino acid in blood has ceased to decrease or has increased, the prognosis of the subject is determined to be improvement of virus infection disease.

### [Aspect 15]

The method according to any one of Aspects 12 to 14, wherein when the indicator associated with a D-amino acid indicates that the amount of a D-amino acid in blood has returned to within a criterion range, it is determined that the amount of virus in the subject has decreased to a healing level.

### [Aspect 16]

The method according to any one of Aspects 12 to 15, wherein when the indicator associated with a D-amino acid indicates that the amount of a D-amino acid in blood has returned to within a criterion range, the prognosis of the subject is determined to be the healing of virus infection disease.

### [Aspect 17]

The method according to any one of Aspects 9 to 16, wherein the indicator associated with a D-amino acid is a measurement value for the D-amino acid in blood or its correction value or correction formula.

### [Aspect 18]

The method according to any one of Aspects 9 to 17, wherein the D-amino acid is one or more D-amino acids selected from the group consisting of D-proline, D-serine, D-alanine, and D-asparagine.

### [Aspect 19]

The method according to any one of Aspects 9 to 18, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

### [Aspect 20]

A composition for modulating the amount of virus in a subject, comprising:

an agent for modulating an indicator associated with a D-amino acid in the subject.

### [Aspect 21]

The composition according to Aspect 20, wherein the indicator associated with a D-amino acid is a measurement value for the D-amino acid in blood or its correction value or correction formula.

### [Aspect 22]

The composition according to Aspect 20 or 21, wherein the agent for modulating the indicator associated with a D-amino acid in the subject is a D-amino acid.

### [Aspect 23]

The composition according to any one of Aspects 20 to 22, wherein the D-amino acid is one or more D-amino acids selected from the group consisting of D-proline, D-serine, D-alanine, and D-asparagine.

### [Aspect 24]

The composition according to any one of Aspects 20 to 23, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

### [Aspect 25]

The composition according to any one of Aspects 20 to 24, for treating or preventing virus infection in the subject by modulating an indicator that is associated with a D-amino acid in the subject and shifts with virus infection.

### [Aspect 26]

The composition according to Aspect 25, wherein the shift in the indicator associated with a D-amino acid in the subject due to virus infection is a shift indicating a decrease in the amount of a D-amino acid in blood.

### [Aspect 27]

The composition according to Aspect 25 or 26, wherein the modulating of the indicator associated with a D-amino acid in the subject includes supplementing the D-amino acid in blood which decreased due to virus infection.

### [Aspect 28]

A system for carrying out the method according to any one of Aspects 9 to 19, comprising:

an input unit for inputting information from a subject;

an analytical measurement unit for analyzing and/or measuring the information from the subject inputted via the input unit to obtain an indicator associated with a D-amino acid in the subject;

a memory unit for storing a determination value associated with virus infection and/or virus infection disease;

a data processing unit for processing the indicator obtained by the analytical measurement unit for the subject based on the determination value stored by the memory unit to make a determination on the amount of virus in the subject and/or the prognosis of virus infection disease in the subject; and the output unit for outputting the results of determination by the data processing unit as information about the amount of virus in the subject and/or the prognosis of virus infection disease in the subject.

An embodiment of the present invention makes it possible to control the amount of virus in a subject and efficiently treat or prevent virus infection disease by modulating an indicator related to D-amino acids in the subject that has shifted, or may shift in the future, with viral infection.

An embodiment of the present invention makes it possible to efficiently estimate the amount of virus in a subject and efficiently predict the prognosis of virus infection disease in a subject by making a determination using an indicator associated with a D-amino acid in the subject.

### BRIEF DESCRIPTION OF THE DRAWINGS

FIG. 1 is a schematic diagram showing the experimental protocol for D-alanine administration to model mice infected with influenza virus in Example 1.

FIG. 2 indicates a graph showing the rates of change in body weights of model mice infected with influenza virus and received D-alanine administration in Example 1.

FIG. 3A is a graph showing the results of viral plaque quantification assay of lung tissue on day 5 after D-Ala administration to the Mock, IAV, and IAV+D-Ala groups in Example 1. FIG. 3B is a graph analyzing the correlation between BD-Ala and the amount of virus in the IAV+D-Ala group in Example 1.

FIG. 4 is a graph showing survival rate curves of model mice infected with influenza virus in relation to D-alanine administration in Example 1.

FIG. 5 is a graph showing the correlation between the body weights and the amounts of D-amino acids in the blood of model mice infected with influenza virus for D-alanine administration in Example 1.

FIG. 6-1] FIG. 6-1 is a graph showing BD-Ala, BD-Ser, BD-Asn, and BD-Pro at 48-72 hours post infection for Mock (−) and IAV (+) groups in Example 1.

FIG. 6-2] FIG. 6-2 is a graph showing B % D-Ala, B % D-Ser, B % D-Asn, and B % D-Pro at 48-72 hours post infection for Mock (−) and IAV (+) groups in Example 1.

FIG. 7 is a graph showing the rates of change in body weights of model mice infected with influenza virus in Example 1.

FIG. 8 shows the survival curves of model mice infected with influenza virus and received D-serine or D-alanine administration in Example 1.

FIG. 9 is a graph showing the rates of change in body weights of model mice infected with influenza virus and received D-serine or D-alanine administration in Example 1.

FIG. 10 is a schematic diagram showing the experimental protocol using model mice infected with coronavirus in Example 2.

FIG. 11-1] FIG. 11-1 is a graph showing the changes over time in BD-Ala, BD-Ser, BD-Pro, and BD-Asn of model mice infected with coronavirus in Example 2.

FIG. 11-2] FIG. 11-2 is a graph showing the changes over time in B % D-Ala, B % D-Ser, B % D-Pro, and B % D-Asn of model mice infected with coronavirus in Example 2.

FIG. 12 is a schematic diagram showing the experimental schedule of D-alanine administration in model mice infected with coronavirus in Example 2.

FIG. 13 is a graph showing the doses of D-alanine and the rates of change in body weights in the group of model mice infected with coronavirus in Example 2.

FIG. 14 shows the survival rate curves of model mice infected with coronavirus and received D-alanine administration in Example 2.

FIG. 15-1] FIG. 15-1 is a graph showing the changes over time in BD-Ala, BD-Ser, BD-Asn, and BD-Pro of the SCV(Vehicle) and SCV+D-Ala groups in Example 2.

FIG. 15-2] FIG. 15-2 is a graph showing the changes over time in B % D-Ala, B % D-Ser, B % D-Asn, and B % D-Pro of the SCV(Vehicle) and SCV+D-Ala groups in Example 2.

FIG. 16-1] FIG. 16-1 is a graph showing BD-Ala, BD-Ser, BD-Asn, and BD-Pro for the two groups classified based on 90% weight maintenance (10% weight loss) in the SCV+D-Ala group in Example 2 (-: group that maintained 90% or more weight, +: group that lost 10% or more weight).

FIG. 16-2] FIG. 16-2 is a graph showing B % D-Ala, B % D-Ser, B % D-Asn, and B % D-Pro for the two groups classified based on 90% weight maintenance (10% weight loss) in the SCV+D-Ala group in Example 2 (-: group that maintained 90% or more weight, +: group that lost 10% or more weight).

FIG. 17 is a graph showing the rates of change over time in body weights of model mice infected with coronavirus and received D-alanine administration in Example 2.

FIG. 18 shows the survival curves of model mice infected with coronavirus and received D-alanine administration with weight loss and death as composite endpoints in Example 2.

FIG. 19 shows the survival curves of model mice infected with coronavirus classified for the amount of D-alanine in the blood in Example 2.

FIG. 20 is a block diagram schematically showing an exemplary configuration of the system of the present invention.

FIG. 21 is a schematic flowchart showing an exemplary processing carried out by the system of the present invention (the method of the present invention).

### DETAILED DESCRIPTION

The present invention is described hereinafter in detail with reference to specific embodiments thereof. However, the present invention is not limited to the following embodiments and can be carried out in any embodiment that does not deviate from the gist according to the present invention.

All patent publications, patent application publications, and non-patent documents cited in this disclosure are incorporated herein by reference in their entirety for all purposes.

In the following description, “the first method of the present invention,” “the second method of the present invention,” and “the third method of the present invention” described below may be referred to collectively as “the methods of the present invention” as appropriate.

### [Definitions]

Amino acids and residues thereof may herein be represented by three-letter abbreviations well known to a person skilled in the art. The three-letter abbreviations of major amino acids are shown in the following table.

TABLE 1

| Abbreviation | Definition |
|---|---|
| Ala | Alanine |
| Arg | Arginine |
| Asn | Asparagine |
| Asp | Aspartic acid |
| Cys | Cysteine |
| Gln | Glutamine |
| Glu | Glutamic acid |
| Gly | Glycine |
| His | Histidine |
| Ile | Isoleucine |
| Leu | Leucine |
| Lys | Lysine |
| Met | Methionine |
| Phe | Phenylalanine |
| Pro | Proline |
| Ser | Serine |
| Thr | Threonine |
| Trp | Tryptophan |
| Tyr | Tyrosine |
| Val | Valine |
| D-AA | D-amino acid |
| L-AA | L-amino acid |

### *D-Amino Acid *L-Amino Acid

The terms “D-amino acids” (abbreviated herein as “D-isomer”) and “L-amino acids” (as “L-isomers”) refer to stereoisomers of amino acids based on the D/L notation of IUPAC nomenclature. The D-isomer and the L-isomer are enantiomers to each other. It is known that the majority of protein-constituent amino acids existing in vivo are L-isomers. Although glycine does not have D- and L-isomers, glycine is treated herein as D-isomer for convenience unless otherwise specified.

Specific examples of D-amino acids herein include, although are not limited to, glycine, D-alanine, D-histidine, D-isoleucine, D-allo-isoleucine, D-leucine, D-lysine, D-methionine, D-phenylalanine, D-threonine, D-allo-threonine, D-tryptophan, D-valine, D-arginine, D-cysteine, D-glutamine, D-proline, D-tyrosine, D-aspartic acid, D-asparagine, D-glutamic acid, and D-serine. Preferred among them are neutral aliphatic amino acid, glycogenic amino acids related to energy metabolism and glucose metabolism pathways, and amino acids which are relatively abundant in blood, e.g., D-proline, D-serine, D-alanine, and D-asparagine. These D-amino acids may be used either singly or in combination of any two or more at any ratios.

D-cysteine in a biological sample is oxidized to D-cystine ex vivo. Therefore, an indicator (e.g., amount) related to D-cysteine contained in a biological sample can be calculated by measuring an indicator (e.g., amount) related to D-cystine in the sample instead of D-cysteine.

*Indicator Associated with D-Amino Acids

The term “indicator associated with a D-amino acid” herein refers to an indicator that is obtained from a living organism and has some relationship to a D-amino acid (in this connection, a measurement or test value of an indicator associated with a D-amino acid obtained from the subject may also be abbreviated simply as, e.g., a “test value of D-amino acid”). Examples of indicators associated with D-amino acids include measurement values for D-amino acids in blood or their correction values or correction formulae.

An example of a measurement value for an indicator associated with a D-amino acid in blood is the amount of the D-amino acid in blood. The term “the amount of a D-amino acid in a blood” herein refers to the amount of the D-amino acid contained in the specific blood. The amounts of D-amino acids in blood may be expressed as concentrations. The amounts of D-amino acids in blood may be measured as the amounts in a sample of collected blood that has undergone centrifugation, sedimentation separation, or pre-treatments for analysis. Accordingly, the amounts of D-amino acids in blood can be measured as the amounts of D-amino acids in a blood sample derived from blood, such as collected whole blood, serum, or plasma. For example, in the case of analysis using HPLC, the amount of a D-amino acid contained in a given volume of blood is represented by a chromatogram, and can be quantified by comparison with a standard or analysis by calibration with respect to peak height, area, and shape.

The correction value or formula of a measurement value associated with the amount of a D-amino acid in blood can be any value or formula obtained via correction processing of the amount of the D-amino acid in blood. Specific examples include corrected D/L ratios of the blood D-amino acid level, % D ({(D-amino acid)/(D-amino acid+L-amino acid)}×100), which represents the ratio of the D-isomer of a specific amino acid, D-amino acid clearance, D-amino acid excretion rate (Non-Patent Literatures 3 and 4), formulas and values obtained by using the amount of the D-amino acid as explanatory variables and correcting it according to the purpose, and values obtained via predetermined formulae, etc.

Another example of a correction value for the amount of a D-amino acid is a value obtained by correcting the D-amino acid level for physiological variables, such as age, gender, BMI, and other factors. When the dynamics of a D-amino acid is affected by kidney functions, the D-amino acid level may be corrected with an indicator for kidney functions and the thus-corrected value may be used. Examples of such indicators for kidney functions include, although are not intended to be limited to, one or more selected from creatinine, cystatin C, inulin clearance, creatinine clearance, urinary protein, urinary albumin, (32-MG, αl-MG, NAG, L-FABP, NGAL, glomerular filtration rate, estimated glomerular filtration rate (eGFR), renal function measurement values and estimated formulae using D-amino acids (Patent Literatures 2, 3, and 4). Specific examples include the correction to determine the ratio of the blood level of a D-amino acid in blood to the blood level of creatinine {(the amount of the D-amino acid in the blood)/(the amount of creatinine in the blood)}. Furthermore, it is known that in vivo D-amino acid levels may shift in neurodegenerative diseases (e.g., ALS), autoimmune diseases (e.g., multiple sclerosis), and metabolic diseases (e.g., diabetes) (Patent Literature 5 and Non-Patent Literature 5). Therefore, it is possible to correct the blood D-amino acid level with a shift factor or a marker for each disease.

Another example of an indicator associated with a D-amino acid in a subject is a value or formula obtained by correcting the blood level of the D-amino acid in the subject with an indicator associated with a biological substance from the subject (e.g., L-amino acid). Examples of indicators associated with a biological substance for correction include, although are not limited to, the L-amino acid level and the total amino acid level in the subject's blood.

When a value obtained by correcting the level of a D-amino acid with another test value is used as an indicator associated with the D-amino acid, the criteria for detection and/or stage classification of the amount of virus and/or the symptoms and conditions of virus infection disease based on a change (e.g., a decrease or an increase) in the D-amino acid level and the judgment and analysis methods can be set and changed according to the correction details for the D-amino acid level. For example, when a value obtained by correcting the D-amino acid level as an inverse (e.g., [1/(the D-amino acid level)]) or a multiplier is used as an indicator associated with the D-amino acid, the judgment criteria set based on a change (e.g., a decrease or an increase) in the blood D-amino acid level may be set as an inverse (e.g., a decrease in the blood D-amino acid level indicates an increase in the original value and an increase in the blood D-amino acid level indicates a decrease in the original value) or a logarithm.

In the method of the present invention, the indicators associated with D-amino acids (e.g., the level of the D-amino acid in blood, corrected D/L ratio, % D ({(D-amino acid)/(D-amino acid+L-amino acid)}×100), D-amino acid clearance, D-amino acid excretion rate, etc.) may be used either singly or in combination of any two or more. In the latter case, it is possible to subject a sample to a panel testing combining two or more indicators associated with D-amino acids at the same time.

In the method of the present invention, the sample for measuring the indicator associated with a D-amino acid may be one or more samples obtained via a single test or two or more samples obtained in two or more tests. When two or more samples are used, the samples may be obtained either at the same point in time or at two or more different points in time. The types of these samples may be selected as appropriate depending on various embodiments described below.

The amounts of D-amino acids and/or L-amino acids in a sample such as blood may be determined by any method. For example, they can be quantified by chiral column chromatography, enzymatic methods, or immunological methods using monoclonal antibodies that identify optical isomers of amino acids. The amounts of D-amino acids and/or L-amino acids in a sample may be measured using any method known to those skilled in the art. Examples include: chromatography methods and enzymatic methods (Y. Nagata et al., Clinical Science, 73 (1987), 105. Analytical Biochemistry, 150 (1985), 238, A. D'Aniello et al., Comparative Biochemistry and Physiology Part B, 66 (1980), 319. Journal of Neurochemistry, 29 (1977), 1053, A. Berneman et al., Journal of Microbial & Biochemical Technology, 2 (2010), 139, W. G. Gutheil et al., Analytical Biochemistry, 287 (2000), 196, G. Molla et al., Methods in Molecular Biology, 794 (2012), 273, T. Ito et al., Analytical Biochemistry, 371 (2007), 167, etc.); antibody methods (T. Ohgusu et al., Analytical Biochemistry, 357 (2006), 15, etc.), gas chromatography (GC) (H. Hasegawa et al., Journal of Mass Spectrometry, 46 (2011), 502, M. C. Waldhier et al., Analytical and Bioanalytical Chemistry, 394 (2009), 695, A. Hashimoto, T. Nishikawa et al., FEBS Letters, 296 (1992), 33, H. Bruckner and A. Schieber, Biomedical Chromatography, 15 (2001), 166, M. Junge et al., Chirality, 19 (2007), 228, M. C. Waldhier et al., Journal of Chromatography A, 1218 (2011), 4537, etc.), capillary electrophoresis (CE) (H. Miao et al., Analytical Chemistry, 77 (2005), 7190, D. L. Kirschner et al., Analytical Chemistry, 79 (2007), 736, F. Kitagawa, K. Otsuka, Journal of Chromatography B, 879 (2011), 3078, G. Thorsen and J. Bergquist, Journal of Chromatography B, 745 (2000), 389, etc.), and high performance liquid chromatography (HPLC) (N. Nimura and T. Kinoshita, Journal of Chromatography, 352 (1986), 169, A. Hashimoto et al., Journal of Chromatography, 582 (1992), 41, H. Bruckner et al., Journal of Chromatography A, 666 (1994), 259 N. Nimura et al., Analytical Biochemistry, 315 (2003), 262, C. Muller et al., Journal of Chromatography A, 1324 (2014), 109, S. Einarsson et al., Analytical Chemistry, 59 (1987), 1191, E. Okuma and H. Abe, Journal of Chromatography B, 660 (1994), 243, Y. Gogami et al., Journal of Chromatography B, 879 (2011), 3259, Y. Nagata et al., Journal of Chromatography, 575 (1992), 147, S. A. Fuchs et al., Clinical Chemistry, 54 (2008), 1443, D. Gordes et al., Amino Acids, 40 (2011), 553, D. Jin et al., Analytical Biochemistry, 269 (1999), 124, J. Z. Min et al., Journal of Chromatography B, 879 (2011), 3220, T. Sakamoto et al., Analytical and Bioanalytical Chemistry, 408 (2016), 517, W. F. Visser et al., Journal of Chromatography A, 1218 (2011), 7130, Y. Xing et al., Analytical and Bioanalytical Chemistry, 408 (2016), 141, K. Imal et al., Biomedical Chromatography, 9 (1995), 106, T. Fukushima et al., Biomedical Chromatography, 9 (1995), 10, R. J. Reischl et al., Journal of Chromatography A, 1218 (2011), 8379, R. J. Reischl and W. Lindner, Journal of Chromatography A, 1269 (2012), 262, S. Karakawa et al., Journal of Pharmaceutical and Biomedical Analysis, 115 (2015), 123, Hamase K, et al., Chromatography 39 (2018) 147-152, etc.).

The separation analysis system for optical isomers according to the present invention may be based on a combination of two or more separation analyses. As a specific example, the amounts of D-amino acids and/or L-amino acids in a sample can be measured by using a method for analyzing optical isomers characterized by including the steps of: flowing a sample containing components having optical isomers through a first column packing material as a stationary phase together with a first liquid as a mobile phase to separate the components in the sample; holding each of the components from the sample individually in a multi-loop unit; flowing each of the components from the sample individually retained in the multi-loop unit, together with a second liquid as a mobile phase, through a channel to a second column packing material having an optically active center as a stationary phase, to partition the optical isomers contained in each of the components from the sample; and detecting the optical isomers contained in each of the components from the sample (JP 4291628 B). For HPLC analysis, D- and L-amino acids may be derivatized beforehand with fluorescent reagents such as o-phthalaldehyde (OPA) and 4-fluoro-7-nitro-2,1,3-benzoxadiazole (NBD-F), or made into diastereomers with, e.g., N-tert-butyloxycarbonyl-L-cysteine (Boc-L-Cys) (HAMASE, Kenji and ZAIZU, Kiyoshi, Analysis Chemistry, Vol. 53, pp. 677-690 (2004)). Alternatively, the amounts of D-amino acids and/or L-amino acids in a sample can be measured by immunological methods using monoclonal antibodies that identify optical isomers of amino acids, such as monoclonal antibodies that bind specifically to D-amino acids or L-amino acids. When the total amount of the D-isomer and L-isomer of an amino acid is used as an indicator, it is not necessary to analyze the D- and L-isomers of the amino acids separately, but the amino acid can be analyzed without distinguishing the D- and L-isomers. Also in such cases, it can still be separated and quantified by enzymatic or antibody methods, GC, CE, or HPLC.

The amounts of biomolecules and drugs such as D-amino acids, L-amino acids, creatinine, and proteins may be expressed herein not only in terms of mere mass, weight, and amount (mol) of the substance, but also in terms of mass, weight, and amount (mol) of the substance per tissue, cell, organ, or molecular unit or per volume or weight, or in terms of mass, weight, amount (mol), concentration, specific gravity, and density of the substance in liquid such as blood or urine, or any other physical quantity that can be measured.

### *Viruses and Virus Infection Diseases

The term “virus” used herein refers to a microscopic infectious structure whose minimal components are a nucleic acid carrying genetic information and a protein shell covering the nucleic acid, and which uses the cells of other organisms to replicate itself. The viruses to which the present invention is applicable are not limited and may be any viruses as long as they infect humans, examples thereof including viruses that causes an infection disease to the host organism upon infection.

Examples of viruses include, although are not limited to, viruses belonging to the families Orthomyxoviridae (e.g., influenza A virus, influenza B virus, and influenza C virus), Coronaviridae (e.g., SARS coronavirus (SARS-CoV), MERS coronavirus (MERS-CoV), and SARS coronavirus 2 (SARS-CoV-2)), Paramyxoviridae (e.g., measles virus and mumps virus), Rhabdoviridae, Arenaviridae, Bunyavirales, Filoviridae, Retroviridae (e.g., human immunodeficiency virus (HIV)), Togaviridae, Flaviviridae (e.g., hepatitis C virus (HCV)), Picornaviridae (e.g., rhinovirus A, B, and C), Astroviridae, Caliciviridae, Reoviridae, Parvoviridae, Adenoviridae (e.g., human adenovirus A to G), Papillomaviridae, Polyomaviridae, Herpesviridae (e.g., herpes simplex virus 1 (HSV-1), herpes simplex virus 2 (HSV-2), and varicella-zoster virus (VZV)), Hepadnaviridae (e.g., hepatitis B virus (HBV)), and Poxviridae (e.g., variola virus, vaccinia virus, monkeypox virus, and camelpox virus).

The term “infection” as used herein refers to a state in which pathogens such as viruses adsorb, settle, invade, or multiply in host cells, tissues, or organs through various infection routes, and includes apparent infection, inapparent infection, and persistent infection (latent infection). The term “infection disease” as used herein refers to a condition in which infection is established and some symptoms or signs appear (e.g., fever, chills, headache, myalgia, joint pain, etc., in the case of influenza, and pneumonia, etc. in the case of COVID-19). The amount of virus in a subject may be measured by subjecting a collected sample (e.g., pharyngeal secretion, sputum/airway secretion, urine, vaginal secretion, feces, blood, spinal fluid, tissues, cells, organs, etc.) to a test (e.g., biochemical tests, serological tests, endocrine tests, microbiological tests, virological tests, culture tests, microscopic tests, genetic tests (PCR, hybridization, etc.), immunological tests (antigen-antibody detection methods), pathological tests, and imaging tests (endoscopy, contrast medium tests, ultrasonography, CT scan, MRI scan, etc.), and may be expressed not only in terms of mere mass, weight, amount, or quantity of the substance, but also in terms of mass, weight, amount, or quantity of the substance per tissue, cell, organ, molecular unit, cultured colony, or plaque or per volume or weight, or in terms of mass, weight, amount, quantity, concentration, specific gravity, and density of the substance in liquid such as blood or urine, or any other physical quantity that can be measured. It may also be expressed qualitatively in comparison with an arbitrary standard, such as more, less, increased, decreased, etc.

Examples of virus infection disease include, although are not limited to, common cold syndrome, influenza, severe acute respiratory syndrome (SARS), Middle East respiratory syndrome (MERS), novel coronavirus infection disease (COVID-19), epidemic parotitis (mumps), other viral pneumonia, chicken pox, shingles, measles, oral herpes, genital herpes, hepatitis B, hepatitis C, smallpox, and monkeypox.

### *Subjects

The term “subject” herein includes, although are not limited to, vertebrates. Examples of vertebrate include mammals, birds, reptiles, amphibians, and fish. Examples of mammals include humans and non-human mammals such as mice, rats, guinea pigs, monkeys, rabbits, cows, horses, pigs, sheep, goats, camels, dogs, and cats. Examples of birds includes chickens. Among them, the subject may preferably be a human or a non-human mammal, especially a human. In addition, various animals that have had virus infection and/or virus infection disease induced by means of transplantation of virus infection and/or virus infection disease cells, genetic modification, or administration of drugs (DNA, RNA, various vaccines, etc.) may be used as targets. Furthermore, various animal individuals, cells, tissues, organoids, etc. that serve as virus infection disease models may be used as subjects.

*Determination criteria

In the second and third methods of the present invention, as described below, the indicators concerning the D-amino acids of the subject are compared with a predetermined determination criterion to make a determination on, e.g., the estimation of the amount of virus in the subject or the prediction of the prognosis of viral infection in the subject.

The term “determination criterion” as used herein refers to a criterion for making a determination on the estimation of the amount of virus in the subject or the prediction of the prognosis of viral infection in the subject. Examples include, but are not limited to, a predetermined criterion value consisting of a single numerical value (also referred to as the “determination criterion value,” “determination value,” or “clinical decision value” below). or a predetermined reference range defined by an upper and lower limit value (also referred to as the “determination criterion range” below). That is, the term “determination criterion” as used herein encompasses the terms “determination criterion value” and “determination criterion range.” Criterion values (clinical decision values) and criterion ranges can be predetermined by analysis of a subject reference individual or a subject reference population. The reference individual may be a healthy subject and the reference population may be a population of healthy subjects.

The method for comparing the indicator associated with a D-amino acid to a predetermined determination criterion is not particularly limited. When a determination criterion value is used as a determination criterion, the determination criterion value may be used as the upper limit of the indicator associated with the D-amino acid, and the determination may be made based on, e.g., whether the indicator associated with the D-amino acid in the subject is equal to or higher than the determination criterion value or whether it exceeds the determination criterion value. Alternatively, the determination criterion value may be used as the lower limit of the indicator associated with the D-amino acid, and the determination may be made based on, e.g., whether the indicator associated with the D-amino acid in the subject is equal to or lower than the determination criterion value or whether it falls below the determination criterion value. On the other hand, when a determination criterion range is used as a determination criterion, the determination may be made based on, e.g., whether the indicator associated with the D-amino acid in the subject stays within, exceeds, or falls below the judgment criterion range. Alternatively, the determination may be made based on, e.g., whether the indicator associated with the D-amino acid in the subject shifts over time from outside of the determination criterion range to within the determination criterion range, from within the determination criterion range to outside of the determination criterion range, persistently outside of the determination criterion range, or persistently within the determination criterion range, etc.

As the determination criterion for the method of the present invention, a single determination criterion value or a single determination criterion range may be used, or two or more determination criterion values or determination criterion ranges may be used in combination, or one or more determination criterion values and one or more determination criterion ranges may be used in combination.

[Method for Modulating the Amount of Virus in a Subject by Controlling an Indicator Associated with a D-Amino Acid in the Subject, and Method for Treating or Preventing Virus Infection (the First Method of the Present Invention)]

An aspect of the present invention relates to a method for modulating the amount of virus in a subject, including administering an agent for controlling an indicator associated with a D-amino acid in the subject (hereinafter also referred to as the “agent for controlling an agent for controlling the amount of a D-amino acid”) to the subject. Based on the correlation between the amount of virus in a subject and an indicator associated with a D-amino acid in the subject, the amount of virus in the subject is modulated by controlling the indicator associated with a D-amino acid in the subject using an agent for controlling the amount of a D-amino acid. An embodiment includes administering D-alanine and/or D-serine to a subject as an agent for controlling the amount of a D-amino acid to thereby increase the amount of a D-amino acid in the blood of the subject as an indicator associated with a D-amino acid, whereby the amount of virus in the lung of the subject can be decreased. The terms “controlling” and “modulating” include varying an indicator associated with a D-amino acid in a subject toward a target or predetermined value or to within a predetermined criterion range set from a healthy subject or a population of healthy subjects, or varying the amount of virus in a subject toward a target or predetermined value or to within a predetermined criterion range set from a healthy subject or a population of healthy subjects. Real-time monitoring of an indicator associated with a D-amino acid in a subject enables efficient setting of the type, dosage, and administration timing of the agent for controlling the amount of a D-amino acid, which is effective for precise control of the indicator associated with a D-amino acid and contributes to improved accuracy in the adjustment of the amount of virus in the target.

The type of a D-amino acid to be used as an agent for controlling the amount of a D-amino acid is not limited as long as it can control a predetermined indicator associated with a D-amino acid. Preferred D-amino acids include neutral aliphatic amino acids and glycogenic amino acids, which are related to energy metabolism and sugar metabolism pathways. An amino acid may be used singly. or two or more amino acids may be used in combination.

In correlation with an increase in the amount of virus in a subject, there may be cases where the subject develops symptoms and conditions such as fever, headache, sore throat, myalgia, general malaise, chills, gastrointestinal symptoms (such as diarrhea), cough, dyspnea, weight change, or death, or cases where these symptoms and conditions in the subject worsen. A shift in such laboratory values or symptoms and conditions, which can be determined quantitatively and/or qualitatively in a clinical setting, may be used to evaluate adjustments in the amount of virus in the subject. As a specific example, if fever or weight loss persists in a subject with virus infection disease, it can be evaluated that the amount of virus in the subject is maintained and/or increasing, and if administration of an agent for controlling the amount of a D-amino acid leads to inhibition of fever or weight loss in the subject, it can be evaluated that the amount of virus in the subject is decreasing. In addition, such values may be calculated, estimated, or evaluated using correlations, regression equations, etc., with objective and/or explanatory variables for parameters including the amount of virus in the subject, indicators associated with D-amino acids, relevant laboratory values, symptoms, and conditions. When modulating the amount of virus in a subject corresponds to treatment and/or prevention of virus infection disease, then the reduction of, and/or the prevention the increase of, and/or the suppression of the increase rate of, the amount of virus in the subject may be referred to as prevention before the onset of the virus infection and/or virus infection disease, and as treatment after the onset of the virus infection and/or virus infection disease.

The first method of the present invention provides a completely new approach to the treatment or prevention of virus infection disease in a subject, by focusing on the correlation between the amount of virus in the subject and an indicator associated with a D-amino acid (e.g., the amount of a D-amino acid in blood) in the subject, and administering to the subject an ingredient to control and regulate these values. Specifically, it makes it possible to adjust the amount of virus in a subject by controlling an indicator associated with a D-amino acid in the subject by any means.

Agents for controlling an indicator associated with a D-amino acid in a subject (agents for controlling the amount of a D-amino acid) may be, although is not limited to, compositions such as drugs and foods that can increase or decrease the amount of a D-amino acid in cells, tissues, organs, and body fluids by external administration of a D-amino acid, addition or removal of a D-amino acid in foods, and addition or removal of a D-amino acid in culture media. For example, drinking an aqueous solution containing A D-amino acid can increase the concentration of the D-amino acid in the blood, cells and tissues (Non-Patent Literature 6), and ingestion of food from which a D-amino acid is extracted can decrease the concentration of the D-amino acid in the blood. For example, a means of controlling the D-serine level in the kidneys can be used by taking advantage of the fact that D-serine is kidney-directed when administered orally or intravenously (Non-Patent Literature 7). Alternatively, the amount of a D-amino acid may be controlled by altering the metabolic system through the administration of another D-amino acid. As a specific example, D-alanine can be administered to a subject to increase the amount of D-serine in the blood of the subject. The D-amino acids as used herein may include modifications or derivatives of D-amino acids or their pharmaceutically acceptable salts, may contain pharmacologically acceptable carriers, diluents or excipients, or may be in prodrug forms, as long as they can increase or decrease the amount of a D-amino acid in the subject. They may also contain an agent for the treatment of the virus infection disease in the subject. The drug according to the present invention can be formulated by selecting a dosage form suitable for the route of administration. When the drug according to the present invention is used for oral administration, it may be formulated in dosage forms such as tablets, capsules, liquids, powders, granules, and chewables, and when used for parenteral administration, in dosage forms such as injections, powders, and infusion products. These formulations may also contain various auxiliary agents used for pharmaceutical purposes, i.e., carriers and other auxiliaries, such as stabilizers, preservatives, painkillers, flavoring agents, taste modifying agents, emulsifiers, fillers, and pH adjusters. These ingredients may be included to the extent that they do not impair the effect of the drug (composition) according to the present invention. The optical purity of a D-amino acid to be used as a drug or as a raw material may preferably be 50% or higher, more preferably 90% or higher, but any optical purity can be selected without limitation as long as it is within the range of efficacy. However, the amount of the D-amino acid should be designed to allow control of the indicator associated with a D-amino acid as the active ingredient.

Agents for controlling an indicator associated with a D-amino acid in a subject (agents for controlling the amount of a D-amino acid) may be ones that use any physiological mechanism to control the amount of the target D-amino acid. As an embodiment, the amount of the target D-amino acid may be controlled by expression (promotion, inhibition, etc.) or activity (activation, inhibition, stimulation, etc.) of proteins related to absorption, transport, distribution, metabolism (synthesis and degradation), excretion, and action of D-amino acids, such as enzymes (D-amino acid oxidase (DAO), D-aspartate oxidase (DDO), serine isomerase (SRR), etc.), transporters and receptors (N-methyl-D-aspartate type glutamate receptor (NMDAR), etc.). DAO inhibitors increase the amount of a D-amino acid at the site of action by inhibiting D-amino acid oxidation, while inhibitors and activators of D-amino acid transporters increase or decrease the amount of a D-amino acid at the starting point or at the destination. Agents acting on proteins, such as enzymes and transporters, do not have to have a direct effect; for example, they may indirectly change the amount of a D-amino acid or the amount of action by competitive reactions of substrates, agonists and antagonists, or by action through scaffold sharing. Although not intended to be limitative, Non-Patent Literature 8 discloses that the SMCT family, ASCT family, and others expressed in the brain and kidney as D-amino acid transporter proteins can alter the localization of D-amino acids by agonists/inhibitors. Since these transporters are affected by co-transporters (e.g., sodium ions) and by cooperation or competition through scaffolds, the transport activity of a D-amino acid can also be controlled by, e.g., sodium/glucose co-transporter (e.g., SGLT2) inhibitors. In addition, Patent Literature 4 discloses that angiotensin 2 receptor blockers (ARBs) alter the amounts of D-amino acids in the blood. For example, it is possible to screen drugs and candidates that can control an indicator associated with a D-amino acid (e.g., the amount of a D-amino acid) by measuring the amounts of D-amino acids in culture media, cells, tissues, and body fluids before and after administration of drugs for viral infections and evaluating their effects. In such cases, the effect can be evaluated by measuring the amounts of D-amino acids in body fluids, cells, and tissues at the site of action.

When administering an agent for controlling the amount of a D-amino acid, the efficacy may be confirmed, the administration may be continued or discontinued, or the dosage and timing of administration may be determined, by temporarily or continuously monitoring laboratory values, symptoms and pathological conditions related to an indicator associated with a D-amino acid (e.g., the amount of a D-amino acid in blood) and/or virus levels and/or infection in any given sample of the living subject.

The present invention, when applied, can control an indicator associated with a D-amino acid by changing the amount of the D-amino acid in the subject using any physiological mechanism, thereby modulating the amount of virus in the subject and controlling the symptoms and pathological conditions of the virus infection disease. As an embodiment, it is possible to control the amount of a D-amino acid in the organism and thereby modulate the amount of virus by controlling the expression (promotion, inhibition, etc.) and/or activity (activation, inhibition, stimulation, etc.) of proteins related to absorption, transport, distribution, metabolism (synthesis and/or degradation), excretion, or action of D-amino acids, or transporters or receptors for D-amino acids.

Therefore, the agent for controlling the amount of a D-amino acid that may be used in the present invention may be one that directly or indirectly promotes gene expression of proteins associated with absorption, transport, distribution, metabolism or excretion of the D-amino acid. For example, it may be the protein or a vector expressing the protein, or it may be a factor that promotes the activity upstream of the cascade that promotes the expression of the protein or a vector expressing the factor.

Alternatively, the agent for controlling the amount of a D-amino acid that may be used in the present invention may directly or indirectly inhibit gene expression of proteins related to absorption, transport, distribution, metabolism or excretion of D-amino acids. For example, it may be selected from small molecule compounds, aptamers, antibodies, antibody fragments, and antisense RNA or DNA molecules, RNAi-directed nucleic acids, microRNAs (miRNAs), ribozymes, genome-edited nucleic acids and their expression vectors.

### [Method for Estimating the Amount of Virus in a Subject's Organism (the Second Method of the Present Invention)]

An aspect of the present invention relates to a method for estimating the amount of virus in a subject's organism, including making a determination on the amount of virus in the subject's organism using an indicator associated with a D-amino acid in the subject (hereinafter also referred to as “the second method of the present invention”).

It is possible to estimate and determine the amount of virus in a subject from a laboratory value of an indicator associated with a D-amino acid, utilizing the correlation between the amount of virus in the subject and the indicator associated with a D-amino acid in in the subject correlation. This may also be referred to as a test for the amount of virus. As a specific example, when the amount of a D-amino acid in the blood of a subject (an indicator associated with the D-amino acid) is increasing, it can be estimated or determined that the amount of virus in the subject is decreasing, while when the amount of a D-amino acid in the blood is decreasing, it can be estimated or determined that the amount of virus in the subject is increasing. An arbitrary determination criterion value (clinical decision value) or determination criterion range can be set for an indicator associated with a D-amino acid. This allows estimation and determination of the amount of virus or virus infection disease in a subject based on a laboratory value of the indicator associated with the D-amino acid in the subject. If there is a correlation between an indicator associated with a D-amino acid and the amount of virus, by regression analysis using the indicator associated with the D-amino acid for any given subject or population of subjects as the explanatory variable and the amount of virus in the given subject or population of subjects as the objective variable, the following equation (I) can be obtained in advance.

Y=a₁×X₁+a₂×X₂+ . . . +aₙ×Xₙ (I)

(I)
In the formula,
a₁ to an each refer to a constant obtained from the regression analysis,
X₁ to Xₙ each refer to a variable for the indicator associated with a D-amino acid selected by the regression analysis, and
b refers to a constant obtained from the regression analysis.

According to an embodiment, the formula (I) above can be used to estimate and determine or test the amount of virus in the subject to be evaluated.

The term “regression analysis” as used herein refers to a method of estimating an equation showing a relationship between an explanatory variable (also referred to as an “independent variables”) and an objective variable (also referred to as a “dependent variable”) using statistical techniques, such as least squares method, moving average method, regression with kernels, etc. Regression analysis is a well-known technique, and any regression analysis may be employed in the present invention. The regression used in the regression analysis according to the present invention may be linear regression or nonlinear regression (e.g., n^{th} order polynomial regression analysis). The regression used in the present invention may be a single regression or a multiple regression. According to the present invention, an equation for calculating the value Y can be obtained by regression analysis in which the indicator associated with a D-amino acid obtained from any given subject or population of subjects (e.g., the values corrected by the amounts of D- and L-amino acids and, if necessary, additionally by the amount of creatinine, etc.) is the explanatory variable and the amount of virus in the subject is the objective variable. Depending on the regression analysis applied, it can be expressed as a variable of powers that are linear, quadratic, cubic, . . . , or n^{th} order functions (where n is a natural number).

For the amount of virus and the index for any D-amino acid to be used for the regression analysis represented equation (I), the data may preferably have a correlation coefficient R≥0.5, more preferably a correlation coefficient R≥0.6, even more preferably a correlation coefficient R≥0.7, and most preferably a correlation coefficient R≥0.8.

### [Method for Predicting the Prognosis of Virus Infection Disease in a Subject (the Third Method of the Present Invention)]

An aspect of the present invention relates to a method for predicting the prognosis of virus infection disease in a subject, including making a determination on the prognosis of virus infection disease in s subject using an indicator associated with a D-amino acid in the subject (hereinafter also referred to as “the third method of the present invention”).

The term “prediction of prognosis” as used herein refers to the prediction or estimation of the subsequent course or outlook for a disease or treatment. Units such as hours, days, weeks, months, years, etc. may be used to express the prediction of prognosis, and Kaplan-Meier analysis may be used as a representative prognostic tool. Prognosis includes: functional prognosis of organs, etc. (such as pneumonia and renal failure); life prognosis of presumed death; and prognosis of symptoms and conditions such as fever, headache, sore throat, myalgia, general malaise, chills, digestive symptoms (such as diarrhea), cough, dyspnea, weight, viral load, and relapse. Prognosis can be expressed quantitatively in terms of variables (parameters) or units or qualitatively (e.g., worse, more severe, better, etc.) for each evaluation item, and provides important information in the selection of treatment measures. According to one embodiment, information on the prognosis of virus infection disease in a subject can be provided, by comparing an indicator associated with a D-amino acid in a subject with a determination criterion value (clinical decision value) or determination criterion range, which has been determined from the amounts of D-amino acids in the blood of a subject or a population of subjects having virus infection disease with information on prognosis.

According to one embodiment, the method of the present invention can provide information to aid in the selection of therapeutic measures for virus infection disease, using an indicator associated with a D-amino acid in the subject. In relation to the amount of virus within a subject and the response or change in viral infection or viral infection to treatment, using the correlation between the amount of a D-amino acid in the blood of a subject and the amount of virus in the subject, a laboratory value of the indicator associated with a D-amino acid is compared with a determination criterion (criterion range or clinical decision value) predetermined for prognosis, to thereby select the most appropriate means from, or assist in selecting it by prioritizing, e.g., treatment, including medication, oxygen therapy (nasal cannula, HFNC, CPAP, NPPV, etc., respiratory failure), surgical treatment, ventilatory management (mechanical ventilator, etc.), ECMO (extracorporeal membrane oxygenation), hemotherapy (dialysis, plasma exchange, apheresis, etc.), thrombosis control, kidney damage control, symptomatic treatment (antipyretic, antitussive, etc.) and dietary treatment. In addition, during the treatment phase, real-time monitoring of the indicator associated with a D-amino acid in a subject to predict prognosis makes it possible to provide information to assist in selecting treatment measures at the next phase. Furthermore, in the event of a pandemic or bioterrorism, based on prognosis prediction and judgment using the indicator associated with a D-amino acid, it can be used for triage to determine and select the priority level of medical care and treatment for subjects and for risk assessment in each pandemic phase selected from the alert phase, pandemic phase, and transition phase. Antiviral drugs, which are used primarily for drug therapy, are drugs that exhibit the effects of inhibition of viral adsorption and entry into host cells, inhibition of intracellular shedding, nucleic acid synthesis, and protein synthesis, and inhibition of extracellular release of viruses, etc., while oxygen therapy, ventilator use, and ECMO are used for severely ill subjects.

According to one embodiment, based on prognostic predictions or determinations made using the indicator associated with a D-amino acid in a subject, the method of the present invention can provide information to select the most appropriate antiviral or other drugs as means for treatment of viral infection disease or to assist in prioritizing the treatment means. As a specific example, the indicator associated with a D-amino acid in a subject can be used for setting a determination criterion (criterion range or clinical decision value) for the effects, side effects, and adverse reactions of a given drug in advance, and for comparing it with a laboratory value of the subject to examine whether the drug should be administered. In addition, according to one embodiment, the indicator associated with a D-amino acid in a subject can be used for predicting and determining the effects, side effects, and adverse reactions after drug administration to the subject, and for providing information to assist in the continuation or discontinuation of administration, or in the determination of the dosage and timing of administration to the subject. Furthermore, the indicator associated with a D-amino acid in a subject can be used for providing information to assist in screening and/or determining the means to control the value of the indicator associated with a D-amino acid in the subject.

Specific examples of drugs that can be used as a means for treating virus infection disease include, but are not limited to: therapeutic drugs for virus infection disease such as neuraminidase inhibitors (e.g., oseltamivir, zanamivir, peramivir, laninamivir), M2 protein inhibitors (e.g., amantadine), RNA polymerase inhibitors (e.g., favipiravir, molnupiravir), cap-dependent endonuclease inhibitors (e.g., baloxavir, marboxil), anti-herpesviruses (e.g., aciclovir, valaciclovir, famciclovir, amenamevir), anti-cytomegalovirus agents (e.g., ganciclovir, foscarnet, valganciclovir, etc.), anti-hepatitis B virus drugs (e.g., entecavir, tenofovir, lamivudine, adefovir, etc.), anti-hepatitis C virus drugs (e.g., sofosbuvir, ribavirin, ledipasvir, etc.), nucleoside-based reverse transcription inhibitors (e.g., tenofovir, emtricitabine, etc.), non-nucleoside-based reverse transcriptase inhibitors (e.g., rilpivirine, efavirenz, etc.), integrase inhibitors (e.g., elvitegravir, dolutegravir, etc.), protease inhibitors (e.g., darunavir, ritonavir, etc.), CCRS inhibitors (e.g., maraviroc, etc.) antibiotics, Chinese herbal medicines (e.g., kakkon-to (kudzu decoction), shoseiryu-to (minor bluegreen dragon decoction), maou-to (ephedra decoction), etc.), acetaminophen, NSAIDs, antihistamines, immunosuppressants (e.g., steroids, baricitinib, etc.), neutralizing antibody drugs (e.g., remdesivir, casirivimab, imdevimab, sotrovimab, etc.), Janus kinase inhibitors (e.g., baricitinib), RNA synthase inhibitors (e.g., remdesivir), biologically active peptides (e.g., adrenomedullin, etc.), GM-CSF preparations (e.g., sargramostim, etc.), anticoagulants (e.g., heparin, etc.), antiparasitic agents (e.g., ivermectin, etc.), humanized anti-human IL-6 receptor monoclonal antibodies (e.g., tocilizumab, sarilumab, etc.), serine protease inhibitors (e.g., nafamostat, etc.), vaccines (e.g., DNA vaccines, RNA vaccines, attenuated vaccines, adenovirus vector vaccines, etc.), oral replacement fluids, infusions, blood transfusions, etc.

### *Others

The method of the present invention can also be used to provide various other types of information in addition to the above. For example, according to one embodiment, the method of the present invention can be used for providing information on screening for viral infection and/or virus infection disease and information on the diagnosis of their pathologies, based on the determination results regarding the amount of virus and prognosis, which have been made using laboratory value of the indicator associated with a D-amino acid. In addition, according to one embodiment, the method of the present invention can be used for providing information on screening for efficacy, adverse effects, and adverse reactions in drug development, clinical trial decisions, alternative endpoints, etc., based on the determination results regarding the amount of virus and prognosis, which have been made using laboratory value of the indicator associated with a D-amino acid. A plurality of types of information as explained above may be provided individually or simultaneously, depending on the purpose.

[System for Providing Information about the Amount of Virus and/or the Prognosis of Virus Infection Disease (the System of the Present Invention)]

An embodiment of the present invention relates to a system for predicting the prognosis of virus infection disease in a subject and/or deciding the amount of virus in a subject's organism by carrying out the second method of the present invention and/or the third method of the present invention (hereinafter also referred to as “the system of the present invention”).

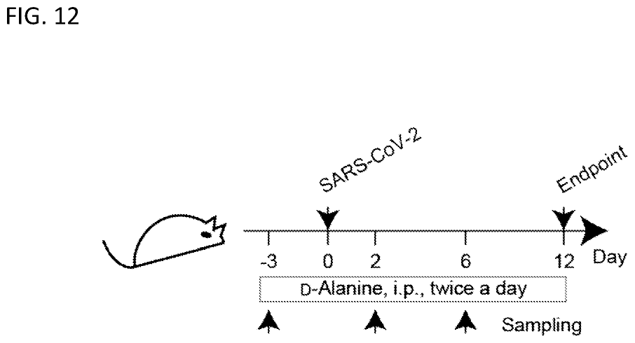

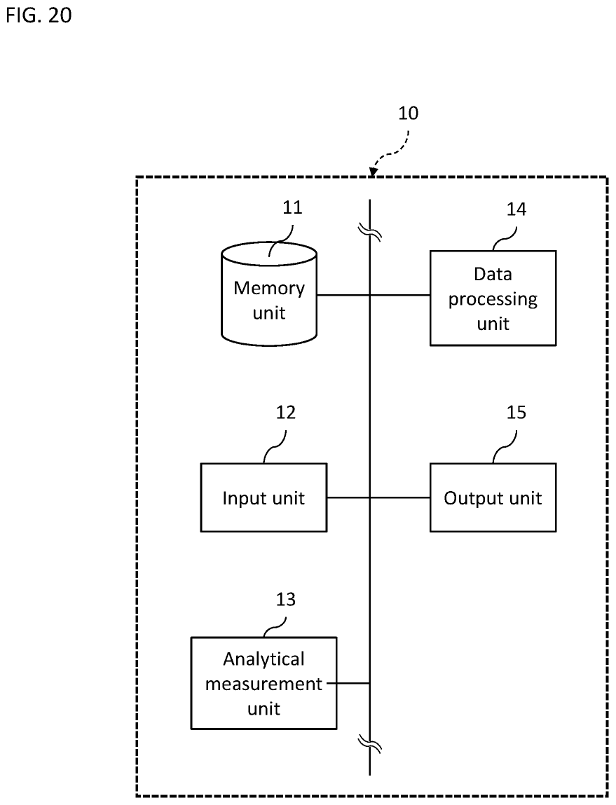

FIG. 20 is a block diagram schematically showing an example configuration of the system of the present invention. However, the configuration shown in FIG. 20 is presented for illustrative purpose only, and the configuration of the system of the present invention is in no way limited by this figure. The sample analysis system 10 shown in FIG. 12 includes a memory unit 11, an input unit 12, an analysis and measurement unit 13, a data processing unit 14, and an output unit 15. The memory unit 11 is configured to store various information including a determination criterion for the amount of virus and/or the prognosis of virus infection disease. The input unit 12 is configured to input various information including data from the subject. The analytical measurement unit 13 is configured to perform various analytical measurements, such as obtaining an indicator associated with a D-amino acid in the subject of the subject by analyzing and measuring data from the subject. The data processing unit 14 is configured to perform various arithmetic processes, such as performing a judgment on the amount of virus and/or the prognosis of virus infection disease of the subject by processing the indicator associated with a D-amino acid of the subject based on the determination criterion. The output unit 15 is configured to output various information, such as information on the amount of virus and/or the prognosis of virus infection disease.

Specifically, the memory unit 11 is composed of memory devices such as RAM, ROM, flash memory, etc., fixed disk devices such as hard disk drives, or portable storage devices such as flexible disks, optical disks, etc. The memory unit 11 is configured to store data and instructions input from the input unit 12, data measured by the analysis and measurement unit 13, results of arithmetic processing performed by the data processing unit 14, and various other information, such as computer programs and databases used in various processes of the information processing device realizing the sample analysis system 10. The computer program may be installed on a computer-readable recording medium, such as a CD-ROM or DVD-ROM, or via the Internet. The computer program is installed in the memory unit 11 using a known setup program or the like.

The input unit 12 is an interface with the outside of the sample analysis system 10, and also includes a keyboard, mouse, and other operating units. This allows the input unit 12 to input data measured in the analysis and measurement section 13, instructions for calculation processing to be performed in the data processing unit 14, and the like. The input unit 12 may also include, for example, an interface section that can input measured data, etc. via a network or a storage medium, in addition to the operation section, if the analytical measurement unit 13 is external.

The analytical measurement unit 13 is configured to obtain an indicator associated with a D-amino acid in the subject of the subject by analyzing and measuring information from the subject. For example, the analytical measurement unit 13 can be configured to measure at least the amount of a D-amino acid from a blood sample of the subject. Accordingly, the analytical measurement unit 13 can be configured to enable separation and measurement of D- and L-isomers of amino acids. Amino acids may be analyzed one at a time, or they may be analyzed collectively for some or all types of amino acids. Although not intended to be limitative, the analytical measurement unit 13 may be a chiral chromatography system, preferably a high-performance liquid chromatography system, equipped with a sample introduction section, an optical partitioning column, and a detection section. In terms of detecting only the amount of a specific amino acid, quantification may be performed by enzymatic or immunological methods. The analytical measurement unit 13 may be configured separately from the system for evaluating laboratory values, and the measured data and other data may be input via input unit 12 using a network or storage medium.

The data processing unit 14 can select information about the amount of virus and/or the prognosis of virus infection disease in the subject by comparing the indicator associated with a D-amino acid measured by the analytical measurement unit 13 with the determination criterion stored in the memory unit. The indicator associated with a D-amino acid may be a formula or value corrected for the amount of a biological substance in the subject (e.g., the amount of a D-amino acid or a test index), or it may be a formula or value corrected for physiological variables such as age, sex, BMI, etc. The data processing unit 14 executes various arithmetic operations on the data measured by the analytical measurement unit 13 and stored in the memory unit 11 according to a program stored in the memory section. The arithmetic processing is performed by the CPU included in the data processing section. This CPU includes functional modules that control the analytical measurement 13, input unit 12, memory unit 11, and output unit 15, and can perform various types of control. Each of these parts may be composed of independent integrated circuits, microprocessors, software, etc.

The output unit 15 is configured to output information about the amount of virus and/or the prognosis of virus infection disease in the subject, which is the result of the arithmetic processing in the data processing unit. The output unit 15 may be a display device such as an LCD display that directly displays the results of the arithmetic processing, a printer or other output means, or an interface section for output to an external storage device or over a network.

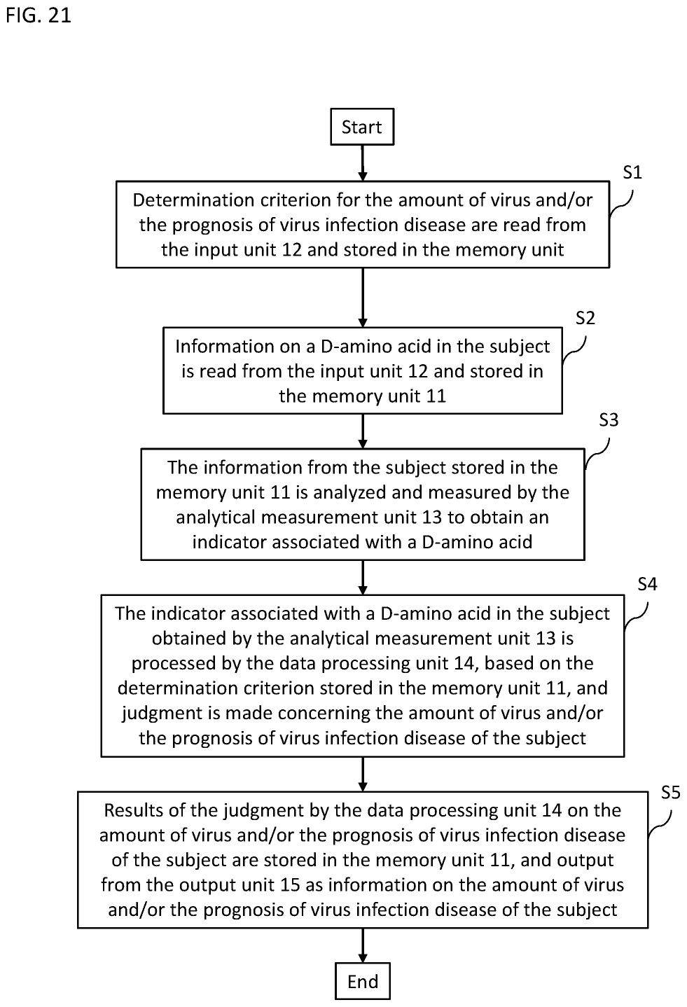

FIG. 21 is a flowchart schematically showing an example of processing by the system (the method of the invention). However, the process shown in FIG. 21 is presented for illustrative purpose only, and the processing by the system of the present invention is in no way limited by this figure. First, a determination criterion for the amount of virus and/or the prognosis of virus infection disease are read from the input unit 12 and stored in the memory unit 11 (step S1). Next, information on a D-amino acid in the subject is read from the input unit 12 and stored in the memory unit 11 (step S2). Then, the information from the subject stored in the memory unit 11 is analyzed and measured by the analytical measurement unit 13 to obtain an indicator associated with a D-amino acid in the subject (step S3). Next, the indicator associated with a D-amino acid in the subject obtained by the analytical measurement unit 13 is processed by the data processing unit 14, based on the determination criterion stored in the memory unit 11, and judgment is made concerning the amount of virus and/or the prognosis of virus infection disease of the subject (step S4). The results of the judgment by the data processing unit 14 on the amount of virus and/or the prognosis of virus infection disease of the subject are then stored in the memory unit 11, and output from the output unit 15 as information on the amount of virus and/or the prognosis of virus infection disease of the subject (step S5).

### [Others]

The present invention has been described in detail in accordance with the specific embodiments above. However, the present invention is not limited to these embodiments, and a person skilled in the art can derive various other inventive concepts from the above description, all of which are within the technical scope of the present invention.

For example, a computer program for realizing the system of the invention and implementing the method of the invention using a general-purpose information processing device is provided (hereinafter also referred to as “the program of the present invention”). Specifically, the program of the present invention can be configured as a program including computer instructions that can be installed in a general-purpose information processing device and executed to cause the information processing device and external devices such as input/output interfaces and analysis devices connected thereto to function as, for example, the sample analysis system 10 shown in FIG. 12, which includes the memory unit 11, input unit 12, analytical measurement unit 13, data processing unit 14, and output unit 15. The program of the present invention can be realized with computer programming knowledge known to those skilled in the art. The program of the present invention and a recording medium such as a CD-ROM containing the program are also included in the technical scope of the present invention.

Since the present invention can be implemented by comparing laboratory values of the indicator associated with a D-amino acid in the subject with a predetermined determination criterion (criterion range or clinical decision value), it can be implemented by persons other than medical doctors, such as clinical examination, health checkup, and data processing companies, analysis systems, and analysis programs, without requiring judgment by a medical doctor, and thus does not fall under so-called medical activity, etc. Specifically, since it provides the determination result on detection and/or stage classification of virus infection and/or virus infection disease in a subject based on the indicator associated with a D-amino acid in the subject, it has extremely high technical utility as a preliminary or auxiliary method that does not replace medical practices such as diagnosis and treatment by a medical doctor, but improves the accuracy and efficiency of such diagnosis and treatment.

### EXAMPLES

The present invention will be described in more detail in the following examples. However, these examples are only shown for convenience of explanation, and the present invention is not limited to these examples in any sense. A person skilled in the art can easily make modifications and changes to the present invention based on the description herein, and all such modifications and changes shall be included in the technical scope of the present invention.

Symbols in the Examples have the following meanings.
D-AA: D-amino acid
L-AA: L-amino acid
BD-AA: D-amino acid concentration in blood (serum or plasma) (nmol/mL or μM)
BL-AA: L-amino acid concentration in blood (serum or plasma) (nmol/mL or μM)
B % D-AA: (BD-AA/BD-AA+BL-AA)×100(%)
Mock: Non-infected mice group (control group)
IAV: Mice group infected with an influenza virus (influenza A virus)
SCV: Mice group infected with a coronavirus (SARS-CoV-2)+
+D-AA: Mice group administered with D-amino acid

All experiments were approved by the Animal Committee of the National Institutes of Biomedical Innovation, Health and Nutrition (NIBIOHN) and conducted in accordance with the guidelines of the Japanese Animal Protection and Control Law.

### Example 1: Influenza Model Mice Study

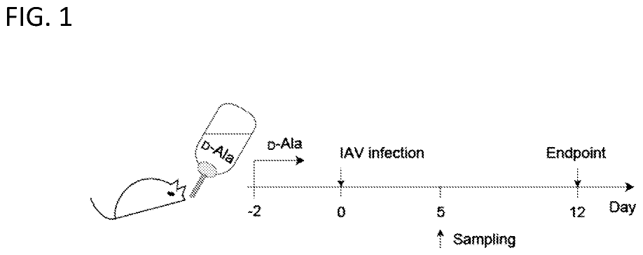

FIG. 1 is a schematic diagram showing the experimental protocol for D-AA administration (+D-AA) to IAV and Mock in Example 1. C57BL6 mice (SLC, Tokyo, Japan) raised in a specific pathogen control facility were used. In order to increase the amount of virus in the mice in a short period of time, 10 L water containing 50 times the TCID50 (median tissue culture infectious dose, 50% infectious dose) of H1N1 influenza A/Puerto Rico/8/34 (PR8 influenza virus strain (ATCC, Manassas, USA) was administered intranasally to anesthetized 4-week-old individuals to infect them. After infection, individuals in each group were weighed and blood samples were collected on a predetermined schedule, and plasma chiral amino acids (D-amino acids and L-amino acids) were quantitatively analyzed by 2D-HPLC.

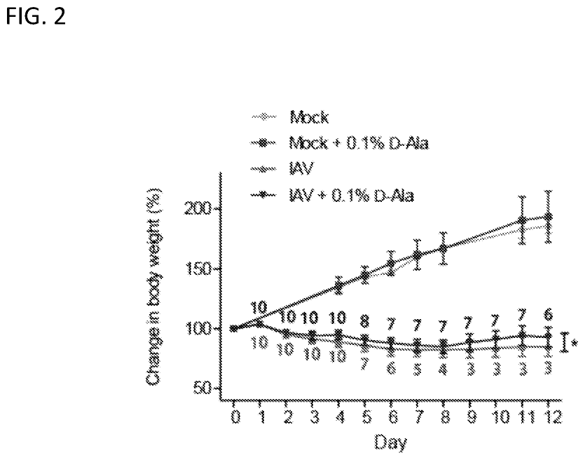

FIG. 2 is a graph showing the rates of change in body weight for the Mock, Mock +D-Ala, IAV, and IAV+D-Ala groups (n=3-10) in Example 1. The numbers in the graph are the number of mice weighed at each time point. Virus-infected mice lost weight, but the rates of weight loss were suppressed in the IAV+D-Ala group (*P<0.05), indicating that administration of D-Ala (a regulator of D-amino acid levels) is resistant and protective against virus infection and increased virus levels The results indicate that administration of D-Ala (a regulator of D-amino acid levels) provided resistance and protection against viral infection and increase in the amount of virus.

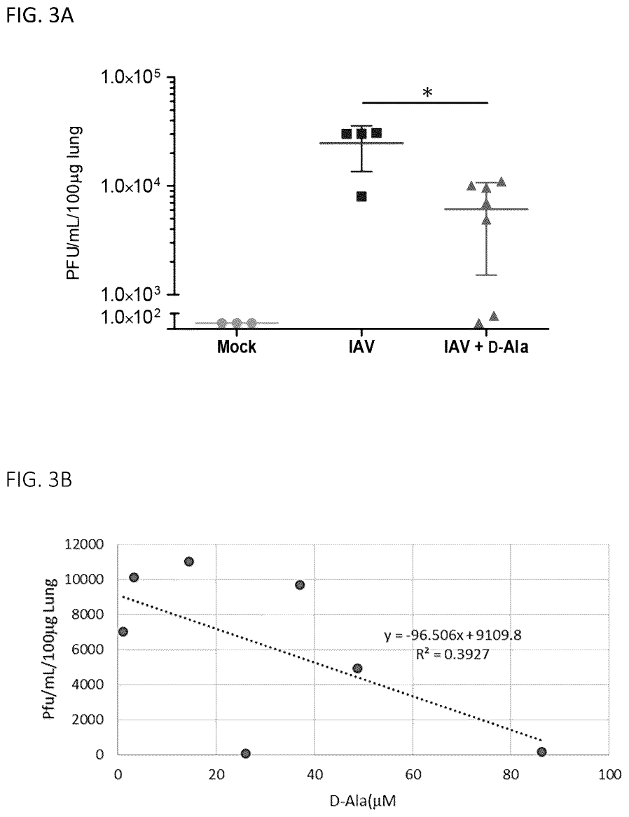

FIG. 3A is a graph showing the results of the viral plaque quantification assay of lung tissue on day 5 after D-Ala administration in the Mock, IAV, and IAV+D-Ala groups (n=3 to 7) in Example 1. Although the amount of virus in the infected mice increased, the increase in the amount of virus in the IAV+D-Ala group was suppressed compared to the IAV group, indicating that administration of D-Ala (an agent for controlling the amount of a D-amino acid) regulated the amount of BD-Ala (an indicator associated with a D-amino acid), whereby the amount of virus in vivo was modulated.

FIG. 3B shows the correlation between BD-Ala and the amount of virus in the IAV+D-Ala group in Example 1. The correlation between BD-Ala and the amount of virus is negative, and when the BD-Ala of a subject is controlled to increase or be at a high level, the amount of virus decreases, while when the BD-Ala of a subject is controlled to decrease or be at a low level, the amount of virus increases. The regression equation obtained from these data was Y (viral load)=−96.5[BD-Ala] +9110, where the correlation coefficient R=0.627. The amount of virus in vivo can be estimated or determined by measuring BD-Ala and substituting it into the regression equation.

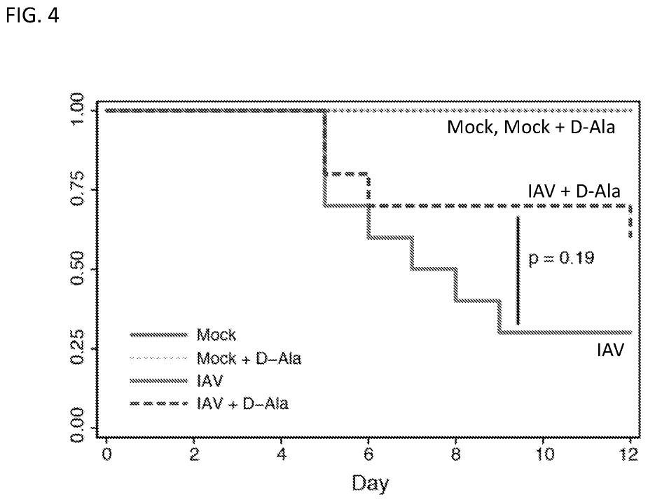

FIG. 4 shows survival curves from Kaplan-Meier analysis with death as the endpoint for the Mock, Mock+D-Ala, IAV, and IAV+D-Ala groups (n=3-10) in Example 1. Severe illness accompanied by an increase in the amount of virus in vivo decreased the survival rate of the influenza virus-infected mice, but administration of D-Ala suppressed the mortality rate, and the prognosis could be changed by controlling the amount of the D-amino acid in the blood of mice and regulating the amount of virus in vivo. The prognosis can be predicted to improve when the amount of D-Ala in the body is elevated, and the selection, dosage, and timing of administration of the agent for controlling the amount of a D-amino acid can be determined as appropriate.

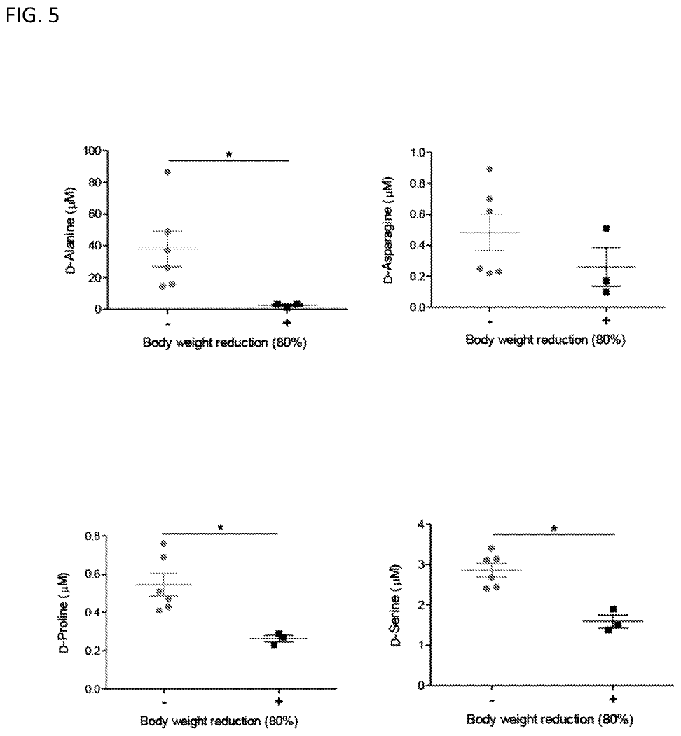

FIG. 5 indicates graphs showing BD-Ala, BD-Asn, BD-Pro, and BD-Ser for the IAV+D-Ala group (n=9) in Example 1, divided into two groups classified based on 80% weight maintenance (20% weight loss) (-: group that maintained 80% or more weight, +: group that lost 20% or more weight). The—group maintained higher BD-AA than the +group, and administration of D-Ala (an agent for controlling the amount of a D-amino acid) help modulate the blood D-amino acid levels and regulate weight changes, a symptom associated with an increase in the amount of virus. BD-AA can be estimated by the degree of weight loss.

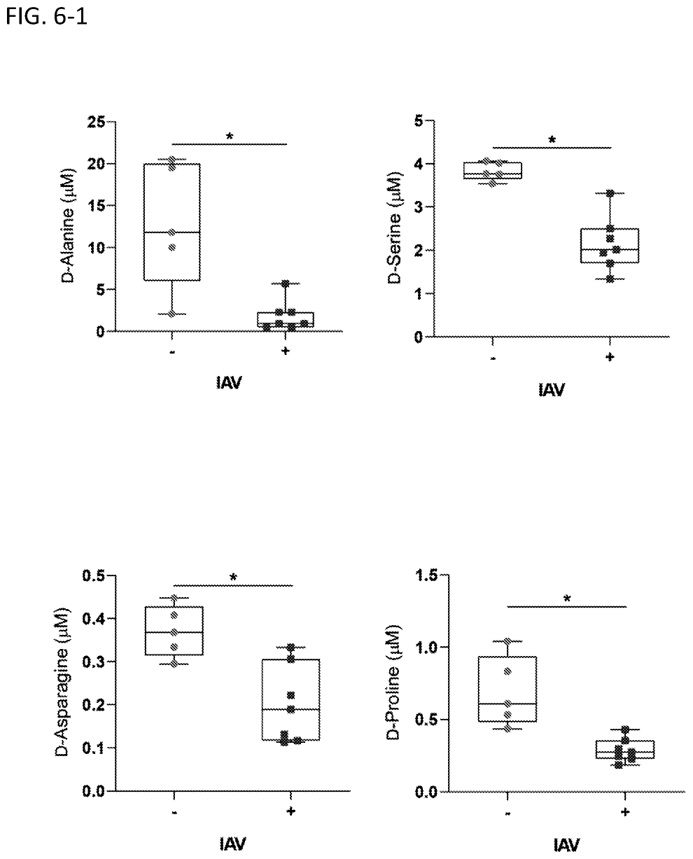

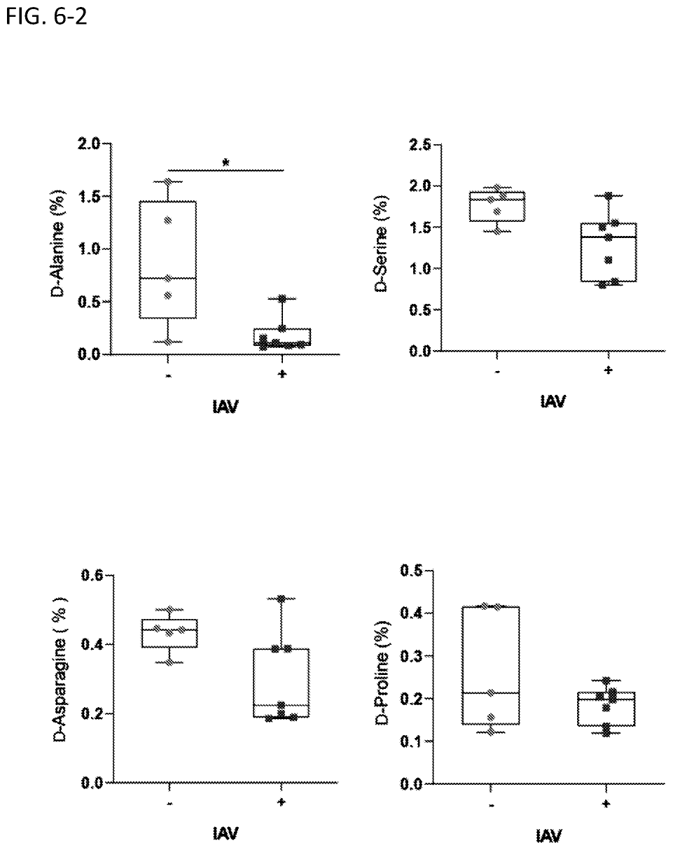

FIGS. 6-1 and 6-2 indicate graphs showing the indicators associated with D-amino acids at 48 to 72 hours post-infection for the Mock (−) and IAV (+) groups in Example 1. Specifically, FIG. 6-1 indicate graphs showing BD-Ala, BD-Ser, BD-Asn, and BD-Pro, and FIG. 6-2 indicate graphs showing B % D-Ala, B % D-Ser, B % D-Asn, and B % D-Pro. As the amount of virus in the IAV (+) group in vivo, BD-AA and B % D-AA, indicators associated with D-amino acids, decreased compared to the Mock (−) group, the reference individual group. Since the indicators associated with D-amino acids can fluctuate with virus infection disease, it is useful to monitor BD-AA as appropriate with the aim of controlling the indicators.

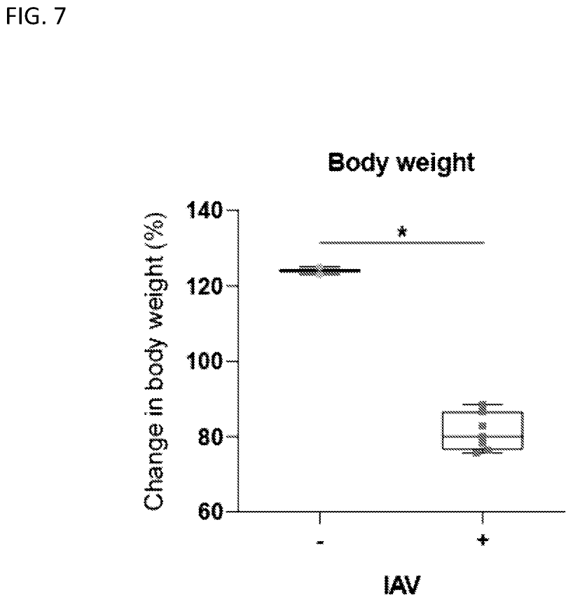

FIG. 7 shows the rates of change in body weight (%) after infection of the Mock (−) and IAV (+) groups in Example 1. The data in FIGS. 6 and 7 show that the increase in the amount of virus in vivo, the symptoms of virus infection disease (weight loss), and the indicator associated with D-amino acids (BD-AA) are related to each other.

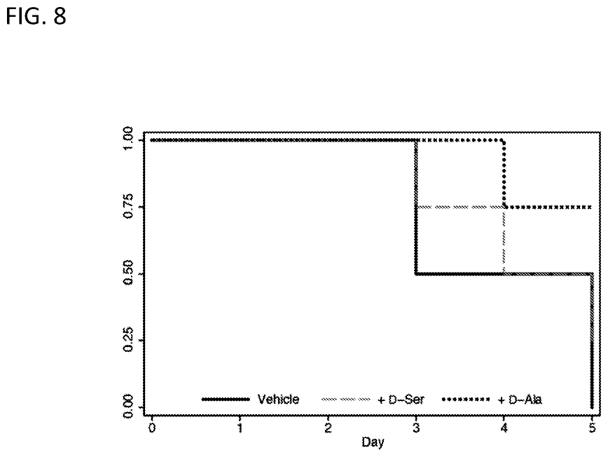

FIG. 8 shows survival curves from Kaplan-Meier analysis with death as the endpoint for the IAV (Vehicle), IAV+D-Ser, and IAV+D-Ala groups in Example 1. While severe illness accompanied by an increase in the amount of virus in vivo reduced the survival rate of the influenza virus-infected mice, administration of D-Ser and D-Ala suppressed the mortality, indicating that the agents for controlling the amounts of D-amino acids help regulate the amount of virus in vivo and alter the life expectancy. It can also be predicted that when the amounts of D-Ser and D-Ala are increased in vivo, the prognosis will improve.

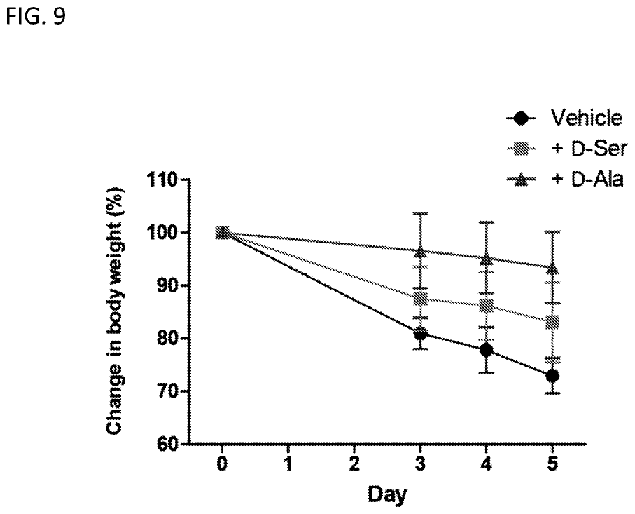

FIG. 9 shows the rates of change in body weight of the IAV (Vehicle), IAV+D-Ser, and IAV+D-Ala groups in Example 1. Although the body weight of the virus-infected groups decreased, the rates of weight loss were suppressed in the IAV+D-Ser and IAV+D-Ala groups, indicating that the administration of D-Ser or D Ala, which are agent for controlling the amounts of D-amino acids, provided resistance and protection against viral infection and increase in the amount of virus.

### Example 2: COVID-19 Model Mice Study

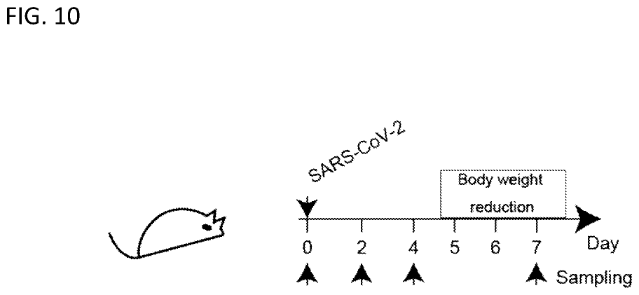

FIG. 10 is a schematic diagram of the experimental protocol using coronavirus-infected mice in Example 2. CAG-hACE2 mice susceptible to SARS-CoV-2, which were developed at the National Institute of Biomedical Innovation, Health and Nutrition, were used. 2×10² to 10⁴ of TCID50 of SARS-CoV-2 was administered to anesthetized 8—to 12-week-old individuals for bronchial infection to create a mouse model of coronavirus infection (Non-Patent Literature 9).

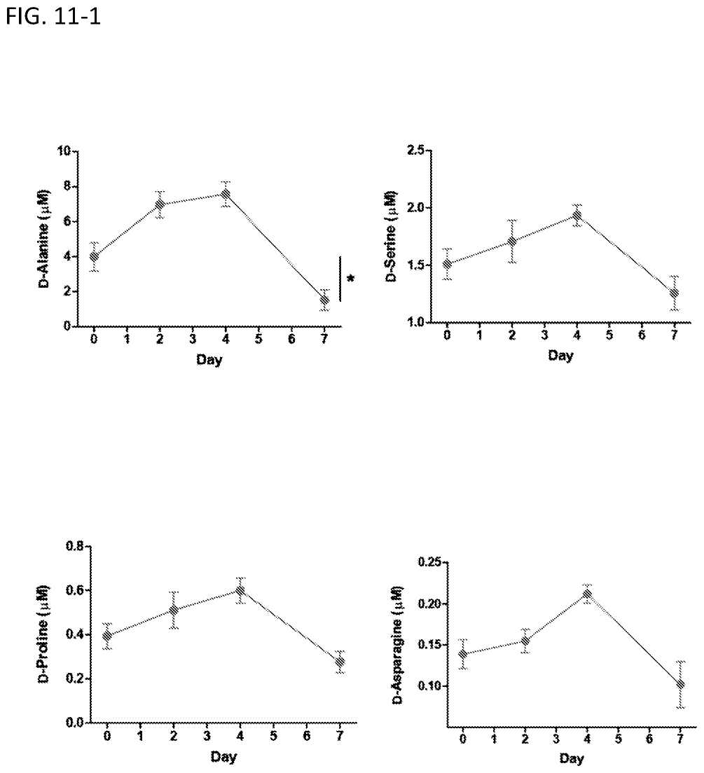

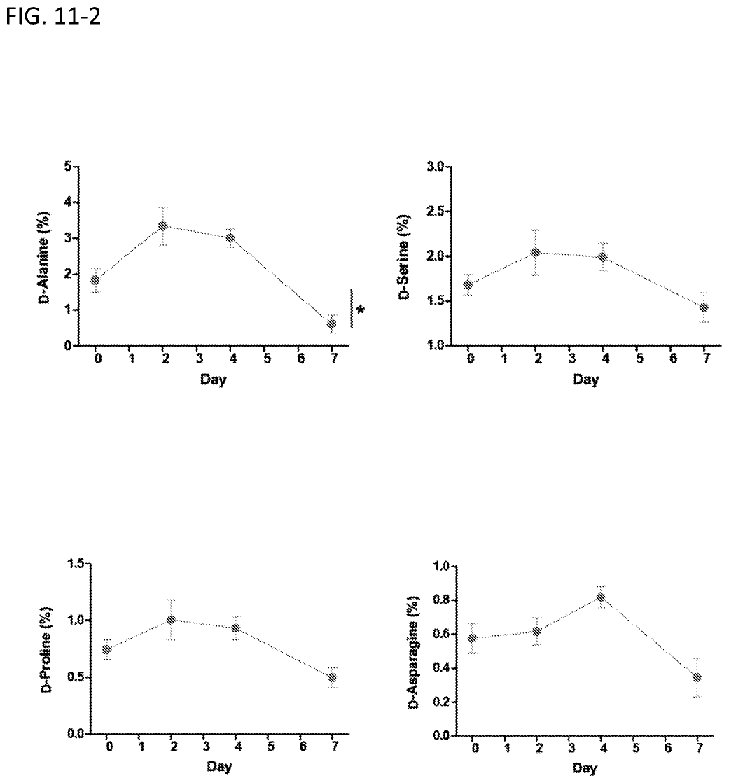

FIGS. 11-1 and 11-2 indicate graphs showing the indices for D-amino acids in the SCV group in Example 2. Specifically, FIG. 11-1 indicate graphs showing changes over time in BD-Ala, BD-Ser, BD-Pro, and BD-Asn, and FIG. 11-2 indicate graphs showing changed over time in B % D-Ala, B % D-Ser, B % D-Pro, and B % D-Asn. Immediately after SARS-CoV-2 infection, both BD-AA and B % D-AA were found to increase due to the supply of D-AA from inside the body, but they decreased with the severity of the disease due to increase in the amount of virus, indicating that the supply of D-AA was insufficient compared to the healthy state. These data show that the indicators associated with D-amino acids can fluctuate with virus infection disease, indicating that it is useful to monitor BD-AA and B % D-AA as appropriate for the purpose of modulating these indicators.

FIG. 12 is a schematic diagram of the experimental schedule of D-Ala administration to coronavirus-infected mice in Example 2. 8—to 12-week-old male and female CAG-hACE2 mice were subjected to intraperitoneal administration with a predetermined dose of D-Ala twice daily, which was stared from 2 days prior to the experiment. The mice were then infected with SARS-CoV-2 via the respiratory tract.

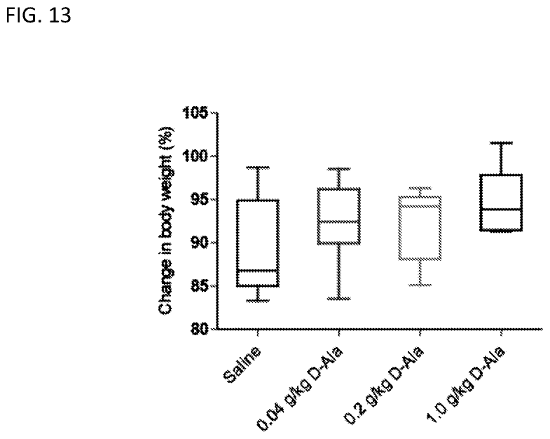

FIG. 13 shows the rates of change in body weights of the SCV (Saline), SCV+D-Ala (doses of 0.04 g, 0.2 g, and 1 g/kg body weight) groups in Example 2. The weight loss, a symptom of increase in the amount of virus, was suppressed in the SCV+D-Ala group compared to the SCV group.

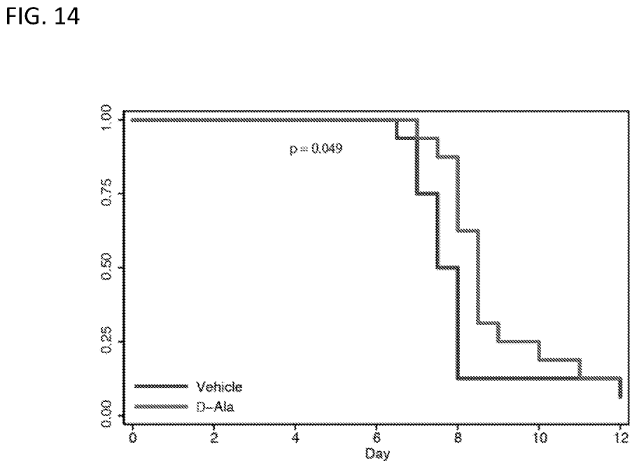

FIG. 14 shows survival curves from Kaplan-Meier analysis with death as the endpoint for the SCV (Vehicle) and SCV+D-Ala groups in Example 2. The survival rate of coronavirus-infected mice was decreased as the amount of virus increased in vivo to make the conditions severer, but administration of D-Ala suppressed the mortality, indicating that controlling the amount of a D-amino acid in the organism of a mouse with an agent for controlling the amount of a D-amino acid helps modulate the amount of virus and alter the life prognosis. It can also be predicted that when the amount of D-Ala in vivo is increased, the prognosis will improve.

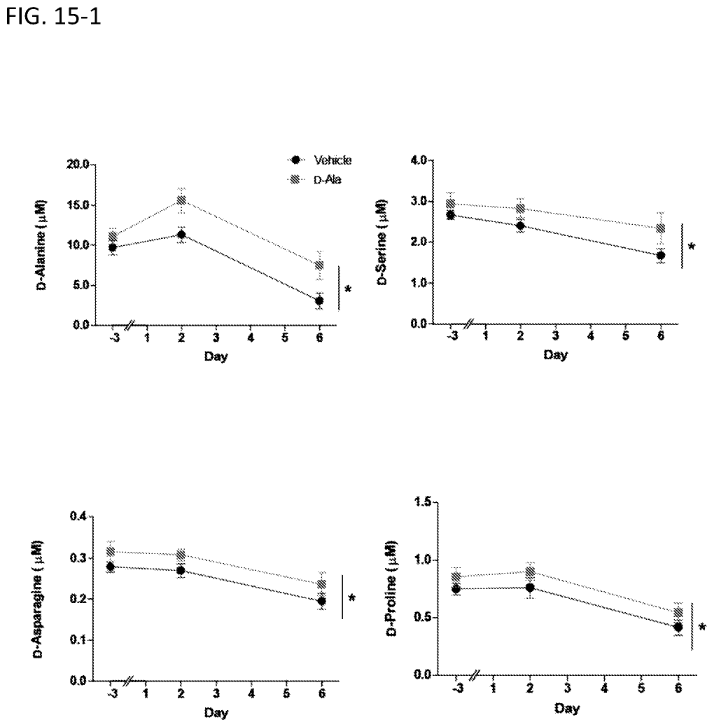

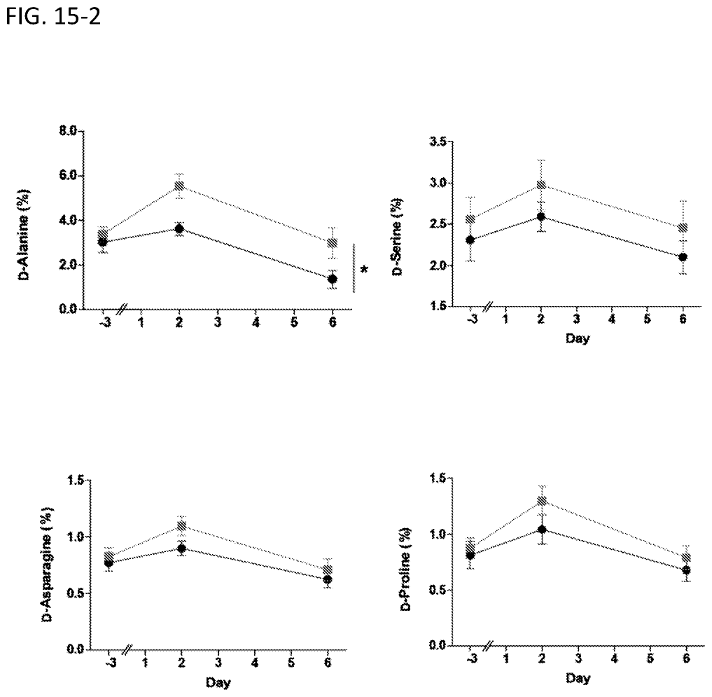

FIGS. 15-1 and 15-2 indicate graphs showing indicators associated with D-amino acids for the SCV (Vehicle) and SCV+D-Ala groups in Example 2. Specifically, FIG. 15-1 indicate graphs showing changes over time of BD-Ala, BD-Ser, BD-Asn, and BD-Pro, and FIG. 15-2 indicate graphs showing changes over time of B % D-Ala, B % D-Ser, B % D-Asn, and B % D-Pro. Administration of D-Ala (an agent for controlling the amount of a D-amino acid) increased BD-AA and B % D-AA, indicating that BD-AA and B % D-AA, indicators associated with D-amino acids, can be controlled.

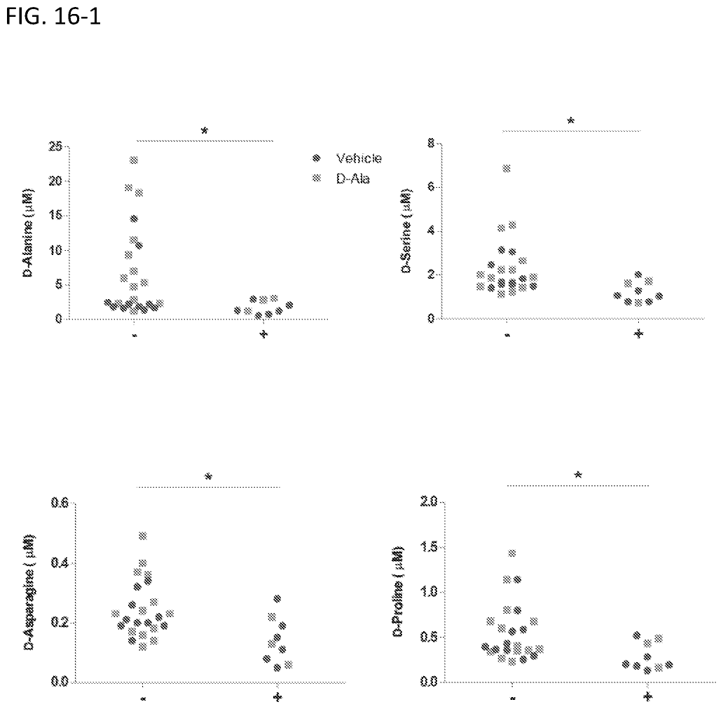

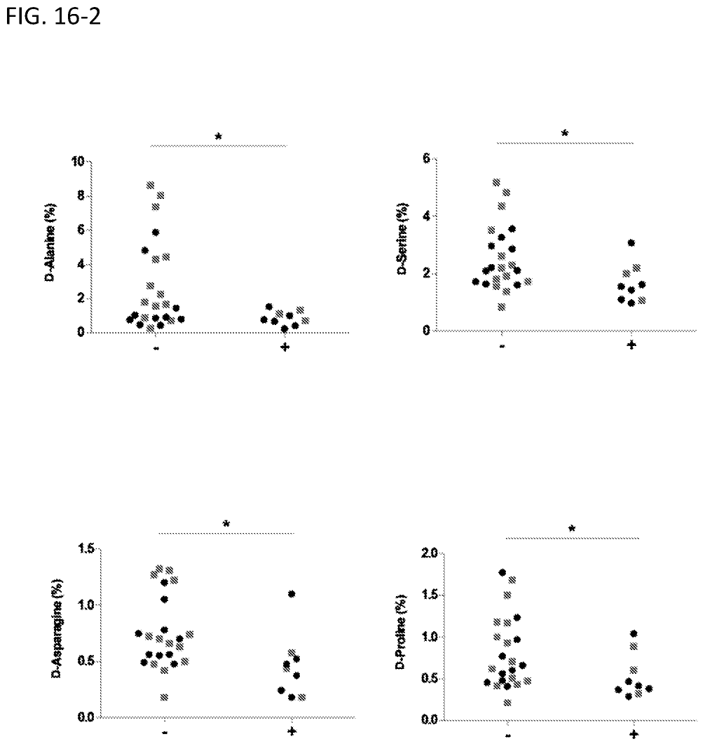

FIGS. 16-1 and 16-2 indicate graphs showing indicators associated with D-amino acids for the SCV+D-Ala group in Example 2, divided into two groups classified based on 90% weight maintenance (10% weight loss) (-: group that maintained 90% or more of body weight, +: group that lost 10% or more of their body weight). Specifically, FIG. 16-1 indicate graphs showing BD-Ala, BD-Ser, BD-Asn, and BD-Pro, and FIG. 16-2 indicate graphs showing B % D-Ala, B % D-Ser, B % D-Asn, and B % D-Pro. The—group maintained higher BD-AA and B % D than the +group, indicating that controlling the amounts of D-amino acids in blood with an agent for controlling the amount of a D-amino acid help modulate the body weight changes associated with an increase in the amount of virus. BD-AA and B % D can be estimated by the degree of weight loss.

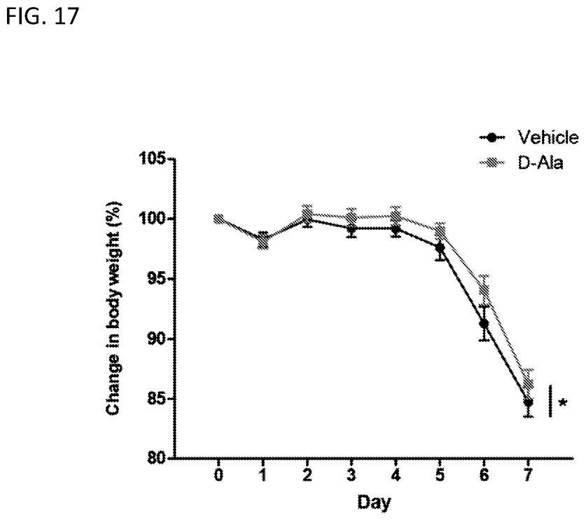

FIG. 17 indicate graphs showing the rates of change in body weights over time for the SCV (Vehicle) and SCV+D-Ala groups in Example 2.-Ala (a regulator of D-amino acid levels) alleviated the symptoms of viral infection. The SCV+D-Ala group had a smaller rate of decrease in body weights than the SCV (Vehicle) group, indicating that administration of D-Ala (an agent for controlling the amount of a D-amino acid) alleviated the symptoms of virus infection disease.

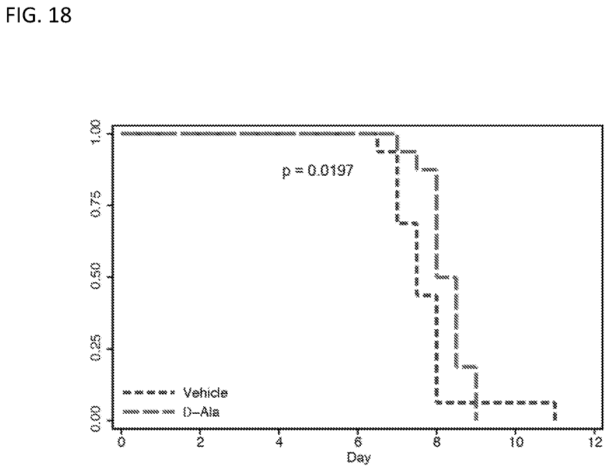

FIG. 18 shows survival curves from Kaplan-Meier analysis for the SCV (Vehicle) and SCV+D-Ala groups in Example 2, with weight loss and death as composite endpoints. While severe illness due to an increase in the amount of virus in vivo reduced the survival rate of coronavirus-infected mice, administration of D-Ala suppressed the weight loss and the mortality, indicating that the agent for controlling the amount of a D-amino acid helps modulate the amount of virus in vivo and alter the prognosis. It can also be predicted that when the amount of D-Ala in vivo is increased, the prognosis will improve.

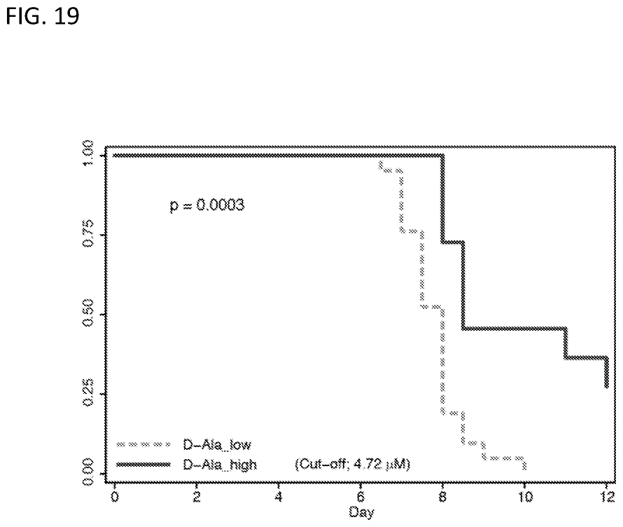

FIG. 19 shows survival curves from Kaplan-Meier analysis with death as the endpoint for the SCV+D-Ala group in Example 2, divided into two groups classified with high (>4.72 μM) and low (<4.72 μM) BD-Ala. When the prognostic threshold is set at 4.72 μM for BD-Ala in vivo, the high BD-Ala group can be predicted to have a better prognosis than the low BD-Ala group.

All of the survival curves discussed above in Example 2 indicate that administration of D-Ala (an agent for controlling the amount of a D-amino acid) had a preventive effect with respect to reduction in the symptoms of virus infection disease.

### INDUSTRIAL APPLICABILITY

The present invention is highly useful in the field of diagnosis and treatment of virus infection and/or virus infection disease.

1. A method for modulating the amount of virus in a subject, comprising:
administering to the subject an agent for controlling an indicator associated with a D-amino acid in the subject.

2. The method according to claim 1, wherein the indicator associated with a D-amino acid is a measurement value for the D-amino acid in blood or its correction value or correction formula.

3. The method according to claim 1, wherein the agent for controlling an indicator associated with a D-amino acid in the subject is a D-amino acid.

4. The method according to claim 1, wherein the D-amino acid is one or more D-amino acids selected from the group consisting of D-proline, D-serine, D-alanine, and D-asparagine.

5. The method according to claim 1, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

6. The method according to claim 1, further comprising treating or preventing virus infection in the subject by modulating an indicator associated with a D-amino acid in the subject which has been shifted due to virus infection.

7. The method according to claim 6, wherein the shift in the indicator associated with a D-amino acid in the subject due to virus infection is a shift indicating a decrease in the amount of the D-amino acid in blood.

8. The method according to claim 6, wherein the modulating of the indicator associated with a D-amino acid in the subject includes supplementing the D-amino acid in blood which has been decreased due to virus infection.

9. A method for estimating the amount of virus in a subject, comprising:
making a determination on the amount of virus in the subject using an indicator associated with a D-amino acid in the subject.

10. The method according to claim 9, wherein when the indicator associated with a D-amino acid indicates that the amount of a D-amino acid in blood has decreased or is equal to or lower than a predetermined threshold, it is determined that the amount of virus in the subject is increasing.

11. The method according to claim 9, wherein the indicator associated with a D-amino acid indicates that the amount of a D-amino acid in blood has ceased to decrease or has increased, it is determined that the amount of virus in the subject has ceased to increase or is decreasing.

12. The method according to claim 9, further comprising:
predicting the prognosis of virus infection disease in the subject based on the results of determination on the amount of virus in the subject.

13. The method according to claim 12, wherein when the indicator associated with a D-amino acid indicates that the amount of a D-amino acid in blood has decreased or is equal to or lower than a predetermined threshold, the prognosis of the subject is determined to be aggravation or increase in severity of virus infection disease.

14. The method according to claim 12, wherein the indicator associated with a D-amino acid indicates that the amount of a D-amino acid in blood has ceased to decrease or has increased, the prognosis of the subject is determined to be improvement of virus infection disease.

15. The method according to claim 12, wherein when the indicator associated with a D-amino acid indicates that the amount of a D-amino acid in blood has returned to within a criterion range, it is determined that the amount of virus in the subject has decreased to a healing level.

16. The method according to claim 12, wherein when the indicator associated with a D-amino acid indicates that the amount of a D-amino acid in blood has returned to within a criterion range, the prognosis of the subject is determined to be the healing of virus infection disease.

17. The method according to claim 9, wherein the indicator associated with a D-amino acid is a measurement value for the D-amino acid in blood or its correction value or correction formula.

18. The method according to claim 9, wherein the D-amino acid is one or more D-amino acids selected from the group consisting of D-proline, D-serine, D-alanine, and D-asparagine.

19. The method according to claim 9, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

20. A composition for modulating the amount of virus in a subject, comprising:
an agent for modulating an indicator associated with a D-amino acid in the subject.

21. The composition according to claim 20, wherein the indicator associated with a D-amino acid is a measurement value for the D-amino acid in blood or its correction value or correction formula.

22. The composition according to claim 20, wherein the agent for modulating the indicator associated with a D-amino acid in the subject is a D-amino acid.

23. The composition according to claim 20, wherein the D-amino acid is one or more D-amino acids selected from the group consisting of D-proline, D-serine, D-alanine, and D-asparagine.

24. The composition according to claim 20, wherein the virus is a virus belonging to a family selected from Orthomyxoviridae, Coronaviridae, Paramyxoviridae, Rhabdoviridae, Arenaviridae, Bunyavirales, Filoviridae, Retroviridae, Togaviridae, Flaviviridae, Picornaviridae, Astroviridae, Caliciviridae, Reoviridae, Parvoviridae, Adenoviridae, Papillomaviridae, Polyomaviridae, Herpesviridae, Hepadnaviridae, and Poxviridae.

25. The composition according to claim 20, for treating or preventing virus infection in the subject by modulating an indicator that is associated with a D-amino acid in the subject and shifts with virus infection.

26. The composition according to claim 25, wherein the shift in the indicator associated with a D-amino acid in the subject due to virus infection is a shift indicating a decrease in the amount of a D-amino acid in blood.

27. The composition according to claim 25, wherein the modulating of the indicator associated with a D-amino acid in the subject includes supplementing the D-amino acid in blood which decreased due to virus infection.

28. A system for carrying out the method according to claim 9, comprising:
an input unit for inputting information from a subject;
an analytical measurement unit for analyzing and/or measuring the information from the subject inputted via the input unit to obtain an indicator associated with a D-amino acid in the subject;
a memory unit for storing a determination value associated with virus infection and/or virus infection disease;
a data processing unit for processing the indicator obtained by the analytical measurement unit for the subject based on the determination value stored by the memory unit to make a determination on the amount of virus in the subject and/or the prognosis of virus infection disease in the subject; and
the output unit for outputting the results of determination by the data processing unit as information about the amount of virus in the subject and/or the prognosis of virus infection disease in the subject.
